(19) United States
(12) Patent Application Publication
(10) Pub. No.: US 2025/0276317 A1
(43) Pub. Date: Sep. 4, 2025

(54) Microfluidic Device and Fabrication Method

(71) Applicant: University of Rochester, Rochester, NY (US)

(72) Inventors: Lisa DeLouise, Rochester, NY (US); Michelle Duan, Rochester, NY (US); Hossein Abolhassani, Rochester, NY (US)

(21) Appl. No.: 19/068,869

(22) Filed: Mar. 3, 2025

Related U.S. Application Data
(60) Provisional application No. 63/708,882, filed on Oct. 18, 2024.
(60) Provisional application No. 63/560,129, filed on Mar. 1, 2024.

(51) Int. Cl. B01L 3/00; B82Y 15/00; B82Y 30/00

(52) U.S. Cl. CPC B01L 3/502753; B01L 3/502707; B01L 2200/027; B01L 2300/0663; B01L 2300/0819; B82Y 15/00; B82Y 30/00

(57) ABSTRACT

A microbubble array comprising a microbubble membrane comprising a plurality of microbubbles, each microbubble of the plurality of microbubbles having a first opening and a second opening diametrically opposing the first opening, and a nanoporous membrane positioned below the microbubble membrane, wherein a first cell type is positioned within at least a portion of the microbubbles of the plurality of microbubbles, and a second cell type is positioned on a bottom surface of the nanoporous membrane. Also disclosed herein are microfluidic devices and microphysiological systems comprising the microbubble array.

### CROSS-REFERENCE TO RELATED APPLICATIONS

This application claims priority to U.S. Provisional Application No. 63/560,129 filed on Mar. 1, 2024, and U.S. Provisional Application No. 63/708,882 filed on Oct. 18, 2024, the contents of which are incorporated by reference herein in their entirety.

### STATEMENT REGARDING FEDERALLY SPONSORED RESEARCH OR DEVELOPMENT

This invention was made with government support under DE027695 awarded by the National Institutes of Health, and 0827862 awarded by the National Science Foundation. The government has certain rights in the invention.

### BACKGROUND OF THE INVENTION

Radiotherapy, widely used for head and neck cancer treatment, may severely damage salivary glands (SGs), and lead to SG dysfunction and xerostomia (i.e., dry mouth). Symptoms may include oral infections, gum disease, tooth decay, or difficulty in speaking, eating, and swallowing. It has been demonstrated that tissue chips can support cell viability and salivary gland tissue mimetic formation in a high-throughput manner with up to a 120-fold reduction in mouse usage in in vivo drug screening experiments. By extracting salivary gland cells from one mouse, up to 40 different drugs may be screened, each with 50 replicates, in a tissue chip that fits in one well of a 96-well plate. Without the use of tissue chips, up to 120 mice would have been needed for the screening of 40 drugs with 3 replicates. The development of the tissue chip model has been a significant improvement but is still not perfect. In general, more than 80% of drugs fail in clinical trial stages and cannot get FDA approval due to the inadequacy of current models. Hence, a more physiologically relevant model is needed for in vitro salivary gland replication. The SG microenvironment is majorly comprised of acinar cells, ductal cells, myoepithelial cells, complex autonomic innervation, vasculature, immune cells, and extracellular matrix (ECM), where neurons stimulate myoepithelial cells and consequently acinar cells to produce saliva which then goes through the ductal structure towards the mouth cavity. Organ-on-a-Chip models can be used for disease modeling and provide 3-dimensional tissue architecture under biorelevant conditions and may enable high throughput drug screening and multi-tissue development with in-situ biosensing. These models have a huge potential to reduce animal use in drug development and screening. In order to further advance the model and replicate in-vivo conditions, relevant tissues must be added to salivary gland tissue chip models to replicate the entire organ, including salivary glands, vasculature and the nerve system. Microbubbles are curvilinear nanoliter cavities formed in polydimethylsiloxane (PDMS) using a patented gas expansion molding (GEM) process (U.S. Pat. Nos. 8,753,880, 9,346,197, and 9,457,497). MB arrays can be used in static cell culture, or they can be mounted into a microfluidic systems or devices where fluid flows in a channel constructed above the MB cavities (Giang, U. B. et al. Biomed Microdevices 16, 55-67 (2014).; Agastin, S. et al. Biomicrofluidics 5, 24110 (2011).; Bobo, B. et al. Lab Chip 14, 3640-3650 (2014).; Chandrasekaran, S. & DeLouise, L. Biomaterials 32, 9316-9327 (2011).; Chandrasekaran, S. et al. Biomaterials 32, 7159-7168 (2011).; Jones, M. C. et al. Biomed Microdevices 15, 453-463 (2013).; Pu, Q. et al. Biomed Microdevices 19, 17 (2017).) However, current systems are limited in their capacity to couple to a second tissue compartment in a microphysiological system (MPS). MPS or so-called organs-on-a-chip may be able to couple many cell types in a device to more appropriately recapitulate a biological system. Previous efforts to study multi-organ interactions were limited to using transwell platforms that require large volumes of liquid such that cell communication is slow and the concentrations of signaling molecules are diluted. Additionally, cell culture is static and lacks any mechanical forces and cell physical stimuli.

Thus, there is a need in the art for methods and devices that overcome the limitations of current MB arrays to improve salivary gland models which include the biorelevant structures to more accurately recapitulate biological systems by offering microliter volumes, perfusion culture, and spatial-temporal control over multi-organ interactions in an array format in a single device.

### SUMMARY OF THE INVENTION

In some aspects, the present invention relates to a microbubble array comprising a microbubble membrane comprising a plurality of microbubbles, each microbubble of the plurality of microbubbles having a first opening and a second opening diametrically opposing the first opening, and a nanoporous membrane positioned below the microbubble membrane, wherein a first cell type is positioned within at least a portion of the microbubbles of the plurality of microbubbles, and a second cell type is positioned on a bottom surface of the nanoporous membrane.

In some embodiments, the first cell type comprises at least one of salivary gland tissue cells, salivary gland cell clusters, or salivary gland cells, and the second cell type comprises endothelial cells. In some embodiments, the microbubble array further comprises at least a third cell type on the bottom surface of the nanoporous membrane, wherein the third cell type comprises at least one of nerve cells or immune cells. In some embodiments, each microbubble of the plurality of microbubbles is at least partially filled with at least one of a polymer, a hydrogel, a polymer crosslinking agent, a hydrogel crosslinking agent. In some embodiments, the microbubble array further comprises a spacing portion with a central opening positioned between the microbubble membrane and nanoporous membrane, wherein a gap with a volume is formed by the opening and the gap is at least partially filled with a polymer or hydrogel.

In some embodiments, the first opening of each microbubble of the plurality of microbubbles is larger than the second opening. In some embodiments, the plurality of microbubbles has a microbubble density of 40 to 30,000 MB/cm². In some embodiments, the microbubble membrane is formed from polydimethylsiloxane (PDMS), and the nanoporous membrane is formed from silicon. In some embodiments, each microbubble of the plurality of microbubbles has a diameter ranging between 20 microns and 2000 microns, and wherein the first opening and the second opening of each microbubble of the plurality of microbubbles has a width ranging between 5 microns and 1000 microns. In some embodiments, the thickness of the microbubble membrane ranges between 0.03 mm and 3 mm, and the thickness of the nanoporous membrane ranges between 50 nm and 500 nm.

In some aspects, the present invention relates to a microfluidic device, comprising: a housing at least partially enclosing at least one microbubble array of claim 1, a top channel in the housing fluidly connected to the first opening of each microbubble of the plurality of microbubbles, and a bottom channel in the housing fluidly connected through the pores of the nanoporous membrane to the second opening of each microbubble in the plurality of microbubbles, wherein one or more fluids may be flowed into the top channel and collected or analyzed from the bottom channel. In some embodiments, the microfluidic device further comprises at least one sensor positioned below the nanoporous membrane in the bottom channel, configured to assess cell secreted factors. In some embodiments, the microfluidic device further comprises at least one of a sensor, optical sensor, and photonic array configured to optically interrogate each microbubble of the plurality of microbubbles. In some embodiments, the microfluidic device further comprises at least one sensor coupled to the microbubble membrane, configured to measure the electrical resistance of the microbubble membrane. In some embodiments, the one or more fluids comprise at least one of water, blood, solutions, solutions of drugs, solutions of therapeutics, solutions of bioactive compounds, buffers, stimulants, or combinations thereof.

In some aspects, the present invention relates to a microphysiological system, comprising: a top portion having a body with at least one cavity, an inlet, and an outlet, each passing through the body, at least one microbubble array of claim 1 positioned within the at least one cavity, a bottom portion comprising a plate having an indentation forming a channel, wherein the top portion is fixedly and removably attached to the bottom portion and the cavity, inlet and outlet are fluidly connected by the channel, and wherein one or more fluids may be flowed into the inlet, analyzed within the channel, and collected from the outlet. In some embodiments, the microbubble membrane is attached to the nanoporous membrane with a spacing portion forming a gap between the microbubble membrane and the nanoporous membrane. In some embodiments, the spacing portion comprises a layer of pressure sensitive adhesive with a central opening passing through the layer, and wherein the gap is at least partially fluidly filled with a hydrogel. In some embodiments, the hydrogel comprises at least one of PEG, Matrigel, collagen, fibrinogen, alginate, polyacrylamide, and hyaluronic acid. In some embodiments, the microphysiological system further comprises one or more sensors positioned within the top or bottom portion of the system, wherein the one or more sensors are selected from the group consisting of: optical sensors, photonic arrays, biosensors, flow rate sensors, fluid pressure sensors, temperature sensors, pH sensors, oxygen sensors, carbon dioxide sensors, and electrochemical sensors. In some embodiments, the one or more fluids comprise at least one of water, blood, solutions, solutions of drugs, solutions of therapeutics, buffers, stimulants, or combinations thereof.

In some embodiments, the microphysiological system is configured to recapitulate one or more disease states, wherein the disease states are selected from tumors, eye diseases, lung diseases, kidney diseases, or stomach diseases.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1A, FIG. 1B, and FIG. 1C depict schematics of an exemplary microbubble (MB) array with two openings bonded to a membrane. In some examples, the MB array with two openings is referred to as a dual opening MB array, a double-side open MB array, or an MB membrane according to aspects of the present invention.

FIG. 2 depicts a schematic of an exemplary MB array bonded to a membrane with a fluid flow across the top openings of the microbubbles.

FIG. 3 depicts a schematic of an exemplary microfluidic device with apical and basal fluid flow paths formed with a disclosed MB array bonded to a membrane with spheroids connected to other cell types according to aspects of the present invention.

FIG. 4 depicts a schematic of an exemplary microfluidic device formed with a disclosed MB array by combining MB arrays and nanomembrane technology with in situ biosensing capability upon optical sensor integration into the device.

FIG. 5A depicts an exploded view schematic of components of an exemplary microphysiological system formed with a disclosed MB array according to aspects of the present invention. FIG. 5B depicts a schematic of a side perspective view of an exemplary microphysiological device. FIG. 5C depicts a schematic of a top perspective view of an exemplary microphysiological device. FIG. 5D depicts an exploded view of components of an exemplary microphysiological device.

FIG. 6 depicts an image of a prototype of an exemplary microphysiological device comprising an MB array disposed within according to aspects of the present invention.

FIG. 7A depicts a flowchart illustrating an exemplary method of fabricating MB arrays according to aspects of the present invention. FIG. 7B depicts a schematic illustrating an exemplary method of fabricating MB arrays using gas expansion molding (GEM) molding.

FIG. 8 depicts a schematic of an exemplary MB array and fluid flow above and into the MB wells.

FIG. 9 depicts an image of an exemplary microfluidic device comprising a MB array under fluid flow.

FIG. 10A depicts a flowchart illustrating an exemplary method of fabricating MB arrays according to aspects of the present invention. FIG. 10B depicts a schematic of an exemplary restricted GEM (rGEM) molding process developed to form or mold MB arrays and/or membranes.

FIG. 11A depicts a flowchart illustrating an exemplary method of fabricating MB arrays. FIG. 11B depicts a schematic of an exemplary inverted GEM (iGEM) molding process developed to form or mold MB arrays and/or membranes.

FIG. 12 depicts a schematic of an exemplary laser ablation process to form cylindrical openings in the microbubbles from the underside of a standard MB array.

FIG. 13 depicts a schematic of an exemplary MB membrane with microbubbles having top and bottom openings according to aspects of the present invention.

FIG. 14A depicts a schematic of an exemplary 2×3 MB array for 3D flow characterization experiments. FIG. 14B depicts a schematic of an exemplary 2×3 MB array for 3D flow characterizations experiments.

FIG. 15A depicts a graph showing the velocity magnitude vs. arc length for the inlet right MB spacing. FIG. 15B depicts a graph showing the velocity magnitude vs. arc length for the outlet right MB spacing.

FIG. 16A depicts a graph of velocity magnitude vs. arc length for the lowest possible inlet right MB spacing. FIG. 16B depicts a graph of velocity magnitude vs. arc length for the lowest possible outlet right MB spacing.

FIG. 17 depicts an exemplary single-side open MB array with scale line and an enlarged cross-sectional view of a single MB with labelled dimension lines.

FIG. 18 depicts Brightfield images of salivary gland mimetic (SGm) formation in MB-chips at days 0, 4, and 7 and corresponding images of fluorescent LIVE (green) and DEAD (red) staining of cells in MB chips at Day 0, Day 7, and Day 14. (scale bars=1 mm, inserts=100 μm).

FIG. 19 depicts a schematic showing an exemplary method of hydrogel encapsulation of SG cluster cells within MB chips.

FIG. 20 depicts a set of images of IHC staining for NKCC1, PIP, IP3R3, and DAPI within SGm at day 14.

FIG. 21 depicts schematics showing an exemplary microfluidic device with single side open MB array (in some examples referred to as a microphysiological system) components and assembly (top), an exemplary microfluidic device comprising MB arrays and dynamic SGm culture (left), and an image depicting an exemplary microfluidic device with SG spheroid culture under perfusion (right).

FIG. 22 depicts a fluid simulation plot of a microfluidic channel with an MB showing the velocity profile of a fluid flow.

FIG. 23A depicts representative images of fluorescent LIVE (green) and DEAD (red) staining of cells in MB chips (e.g., MB array) at Day 7 in static culture. FIG. 23B depicts representative images of fluorescent LIVE (green) and DEAD (red) staining of cells in MB chips at Day 7 in dynamic culture. FIG. 23C depicts a plot showing size distributions of SGm under static culture at day 4. FIG. 23D depicts a plot showing size distributions of SGm under dynamic culture at day 4. FIG. 23E is a plot of qPCR results showing relative Mist1 expression against time for an SGm culture. FIG. 23F is a plot of qPCR results depicting relative K7 mRNA expression against time for an SGm culture. FIG. 23G is a plot of qPCR results depicting relative SMA mRNA expression against time for an SGm culture. FIG. 23H is a plot of qPCR results depicting relative NKCC1 mRNA expression against time for an SGm culture. FIG. 23I is a plot of qPCR results depicting relative Lyz2 mRNA expression against time for an SGm culture.

FIG. 24 depicts an exemplary process of forming the bottom opening of MBs using a vibratome to produce a double-side opening MB array (e.g., an MB membrane).

FIG. 25 depicts a diagram of the development over time of innervated vascularized SGm in a disclosed microphysiological system or device with MB array according to aspects of the present invention.

FIG. 26A depicts representative images of gel presence in the gap of an exemplary microphysiological device. FIG. 26B depicts representative fluorescent microscopy images of gel presence in the gap of an exemplary microphysiological device. FIG. 26C depicts representative images of fluorescent LIVE (green) and DEAD (red) staining of SGm in MB array coupled to the microphysiological device at day 4.

FIG. 26D depicts representative images of fluorescent LIVE (green) and DEAD (red) staining of SGm in MB array coupled to the microphysiological device at day 4.

FIG. 27A depicts a 4× resolution image of an exemplary monolayer cell culture on the underside of the porous membrane at day 4. FIG. 27B depicts a fluorescence microscopy image (4×) HUVECs cultured on the underside of the nanomembrane at day 4 showing live cells (green) and dead cells (red).

FIG. 28A depicts a 10× resolution fluorescence microscopy image of SGm culture in the hybrid device with degradable PEG hydrogel in the gap at day 4. FIG. 28B depicts a 20× resolution fluorescence microscopy image of SGm culture in the hybrid device with degradable PEG hydrogel in the gap at day 4. FIG. 28C depicts a 20× resolution fluorescence microscopy image of SGm culture in the hybrid device with degradable PEG hydrogel in the gap at day 4.

FIG. 29A depicts a schematic of an exemplary MB array with a small second opening. FIG. 29B depicts a schematic of an exemplary MB array with a large second opening.

FIG. 30 depicts an illustrative computer architecture for a computer.

### DETAILED DESCRIPTION

The following discussion omits or only briefly describes conventional features of tissue-on-a-chip and/or organ-on-a-chip devices that are apparent to those skilled in the art. Those of ordinary skill in the pertinent arts may thus recognize that other elements may be desirable and/or necessary to implement the devices, systems, and/or methods described herein. It is noted that various embodiments are described in detail with reference to the drawings. Reference to these various embodiments does not limit the scope of the claims attached hereto. Additionally, any embodiments set forth in this specification are intended to be non-limiting and merely set forth some of the many possible implementations for the appended claims. Further, particular features described herein can be used in combination with other described features in each of the various possible combinations and permutations. As such, it is understood that this detailed description is exemplary and explanatory only and is not restrictive of the broad inventive concepts upon which the embodiments disclosed herein are based.

Unless otherwise specifically defined herein, all terms are to be given their broadest reasonable interpretation. This includes meanings implied from the specification as well as meanings understood by those skilled in the art and/or as defined in dictionaries, treatises, etc.

It is noted that, as used in this specification and the appended claims, the singular forms “a,” “an,” and “the” include plural referents unless otherwise specified. The term “includes” and/or “including,” when used in this specification, specify the presence of stated features, elements, and/or components, but do not preclude the presence or addition of one or more other features, steps, operations, elements, components, and/or groups thereof.

Relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then-described or as shown in the drawing figure under discussion. These relative terms are for convenience of description and normally are not intended to require a particular orientation in actuality. Terms including “inwardly” versus “outwardly,” “longitudinal” versus “lateral,” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. The phrases “operatively” or “operably connected” indicates such an attachment, coupling, or connection that allows the pertinent structures to operate as intended by virtue of that relationship.

Reference throughout the specification to “one embodiment,” “an embodiment,” or “some embodiments” means that a particular feature, structure, or characteristic described in connection with at least one embodiment of the subject matter is included in at least one embodiment of the subject matter disclosed. Thus, the appearance of the phrases “in one embodiment,” “in an embodiment,” or “in some embodiments” in various places throughout the specification is not necessarily referring to the same embodiment. Further, the particular features, structures, or characteristics of “one embodiment,” “an embodiment,” or “some embodiments” may be combined in any suitable manner with each other to form additional embodiments of such combinations. It is intended that embodiments of the disclosed subject matter cover modifications and variations thereof. Terms such as “first,” “second,” “third,” etc., merely identify one of a number of portions, components, steps, operations, functions, and/or points of reference as disclosed herein, and likewise to not necessarily limit embodiments of the present disclosure to any particular configuration or orientation.

Moreover, throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments therebetween. This applies regardless of the breadth of the range. As used herein, the term “about” in reference to a measurable value, such as an amount, a temporal duration, and the like, is meant to encompass the specified value variations of plus or minus 20%, plus or minus 10%, plus or minus 5%, plus or minus 1%, and plus or minus 0.1% of the specified value, as such variations are appropriate and fit within the confines of a functional system.

The terms “proximal,” “distal,” “anterior,” “posterior,” “medial,” “lateral,” “superior,” and “inferior” are defined by their standard usage indicating a directional term of reference. For example, “proximal” refers to a position that is situated nearer to the center of a body or point of attachment or interest. In another example, “anterior” refers to the front of a body or structure, while “posterior” refers to the rear of a body or structure, in relation to a relative viewpoint. In another example, “medial” refers to the direction towards the midline of a body or structure, and “lateral” refers to the direction away from the midline of a body or structure. In some embodiments, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

The terms “patient,” “subject,” “individual,” and the like are used interchangeably herein, and refer to any animal amenable to the systems, devices, and methods described herein. The patient, subject, or individual may be a mammal, and in some instances, a human.

As used herein, “hydrogel” refers to a water-insoluble and water-swellable cross-linked polymer that is capable of absorbing at least 3 times, or at least at least 10 times, its own weight of a liquid. “Hydrogel” can also refer to a “thermo-responsive polymer” as used herein.

As used herein, “microfluidic devices” refers to a chip (e.g, PDMS chip) device using small amounts of fluid to perform laboratory tests.

As used herein “organ-on-a-chip devices” refers to a 3D microfluidic chips, comprising one or more cell cultures, that simulate the activities, mechanics and physiological responses of an organ or organ system under variable biological conditions.

As used herein “microphysiological device” refers to a model of human organs or tissues that simulate the activities, mechanics, and physiological responses of an organ and/or tissue under variable biological conditions.

Single opening microbubble (MB) arrays for culturing cells are lamellar structures that induce streams of fluid into the MBs that recapitulate aspects of fluid dynamics and perfusion of cells in tissue. Cells and/or organoids can be placed within the MBs of the MB arrays, and as fluids flow across the MB openings, a fluid flow is created within the MB where cell byproducts aggregate and may be collected and analyzed. However, single opening MBs limit the fluid flow to only one side of the MBs, and limits coupling to multi-organ microphysiological systems (MPS).

The present invention relates to dual opening MB arrays, also referred to herein as MB membranes, which allow layers of cells, hydrogels, and/or additional flow paths to be formed on the typically closed side of an MB array, creating realistic biomimetic properties that can more accurately represent tissue and multi-organ physiological systems and enable increased nutrient and waste exchange through diffusion and flow on both sides. Such MB membranes, and microfluidic and microphysiological devices comprising these MB membranes disclosed herein, enable more accurate representations of tissue or organ microstructures under relevant biological conditions, thereby allowing for improved tissue and disease recapitulation, enhanced drug screening, and toxicology testing.

Aspects of the present invention relate to an MB array or membrane, also referred to herein as a double-sided open MB array or membrane. In some embodiments, the disclosed MB array or membrane may be integrated with any microfluidic devices, organ-on-a-chip devices, sensors, or microphysiological systems (MPS) known by one of ordinary level of skill in the art. In some embodiments, the disclosed MB array or membrane is capable of coupling with various microfluidic components to make devices and systems that expand the applications of MB technology for scientific applications and drug screening. In some embodiments, the MBs in the MB membrane are capable of culturing tissues, cells, cell cultures, organoids, inducible pluripotent stem cells, or primary tissues positioned above and beneath the MB membrane for improved replication of salivary gland (SG) tissue structure. Aspects of the present invention relate to microfluidic devices and microphysiological systems comprising the disclosed MB array or membrane. Further, aspects of the present invention relate to methods of forming an MB array or membrane.

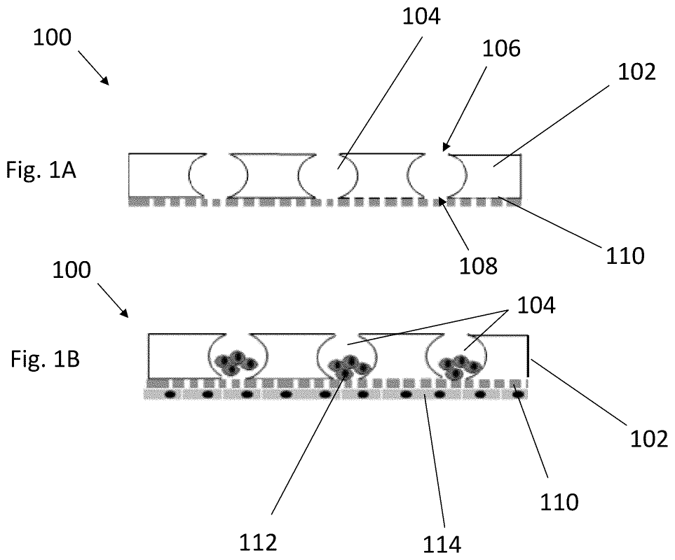

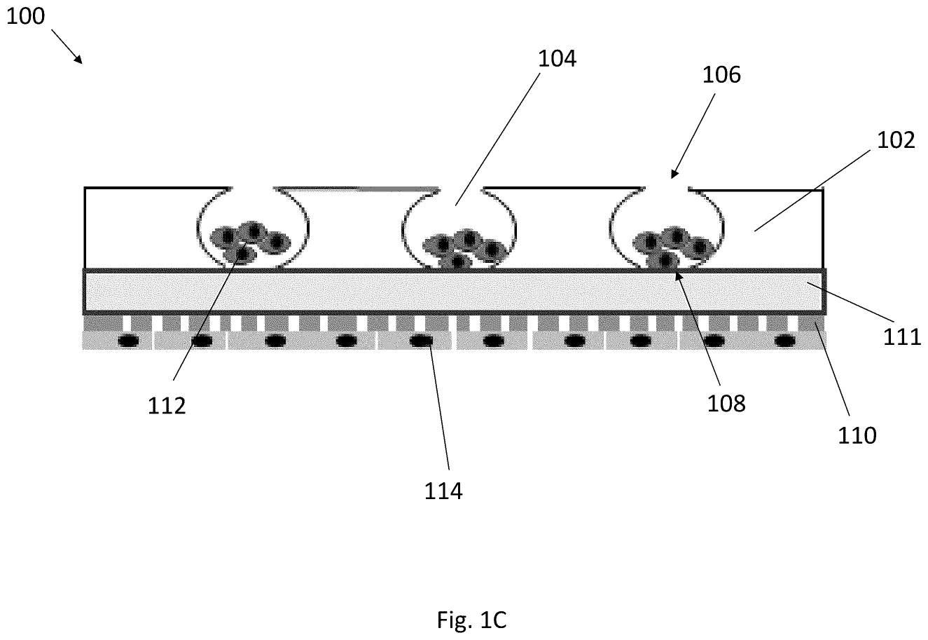

In some aspects, the present invention relates to a double-sided opening MB array, also referred to herein as an MB membrane, a dual opening MB array, or simply an MB array. Referring now to FIGS. 1A and 1B, shown is an exemplary MB array 100 comprising an MB membrane 102 comprising a plurality of microbubbles 104 disposed within the membrane 102. In some embodiments, each microbubble of plurality of microbubbles 104 has a first opening 106 and a second opening 108 diametrically opposing the first opening 106. In some embodiments, the MB array 100 further comprises a nanoporous membrane 110 positioned below the MB membrane 102.

Referring now to FIG. 1C, MB array 100 comprises a spacing portion with a central opening positioned between the microbubble membrane 102 and nanoporous membrane 110, wherein a gap 111 with a volume is formed by the opening of the spacing portion. In some embodiments, the gap 111 is at least partially filled with a polymer or hydrogel. In some embodiments, the spacing portion comprises or is formed by an adhesive layer, or a pressure sensitive adhesive (e.g., pressure sensitive adhesive 316 described herein).

In some embodiments, a first cell type 112 is positioned and/or cultured in at least a portion of the microbubbles of the plurality of microbubbles 104, and a second cell type 114 is positioned below or on a surface of the nanoporous membrane 110. In some embodiments, the MB array 100 may further comprise at least a third cell type positioned and/or cultured in a gel (e.g., a hydrogel) in the gap 111. In some embodiments, the MB array 100 may comprise a fourth cell type, or a fifth cell type. In some embodiments, the MB array 100 may further comprise any number of cell types. It should be appreciated that numerous configurations of cells may be positioned in and/or cultured on various portions of MB array 100. For example, one or more cell types may be positioned in the plurality of microbubbles 104, including patterning or alternating of cells, cell types, or organoids across MB array 100, or within the gap 111 formed by the opening of a spacing portion or adhesive layer. Any number of cells, cell types, organoids, cell clusters, or the like, may be positioned and/or cultured in any configuration on or within MB array 100.

In some embodiments, the plurality of microbubbles 104 comprise between 1-5000 microbubbles, 4-1000 microbubbles, 10-500 microbubbles, 20-200 microbubbles, or 40-100 microbubbles, however the plurality of microbubbles 104 may comprise any number of microbubbles. In some embodiments, the plurality of microbubbles has a microbubble density of 40 to 30,000 MB/cm². In some embodiments, the plurality of microbubbles 104 may have a generally round, circular, elongate and/or spherical shape. In some embodiments, the plurality of microbubbles 104 are curvilinear cavities formed within the membrane 102.

In some embodiments, each microbubble of the plurality of microbubbles 104 may have a diameter of about 20 microns, 50 microns, 75 microns, 100 microns, 125 microns, 150 microns, 175 microns, 200 microns, 225 microns, 250 microns, 275 microns, 300 microns, 325 microns, 350 microns, 375 microns, 400 microns, 425 microns, 450 microns, 475 microns, 500 microns, 525 microns, 550 microns, 575 microns, 600 microns, 625 microns, 650 microns, 675 microns, 700 microns, 725 microns, 750 microns, 775 microns, 800 microns, 825 microns, 850 microns, 875 microns, 900 microns, 925 microns, 950 microns, 975 microns, about 1000 microns, about 1250 microns, about 1500 microns, about 1750 microns, or about 2000 microns. In some embodiments, each microbubble of the plurality of microbubbles 104 may have a diameter ranging between 20 microns and 2000 microns, between 50 microns and 1750 microns, between 60 microns and 1500 microns, between 70 microns and 1250 microns, between 80 microns and 1000 microns, between 90 microns and 900 microns, between 100 microns and 800 microns, between 110 microns and 700 microns, between 120 microns and 600 microns, between 130 microns and 550 microns, between 140 microns and 500 microns, between microns 150 and 450 microns, between 160 microns and 400 microns, between 170 microns and 350 microns, between 180 microns and 300 microns, between 190 microns and 275 microns, or between 200 microns and 250 microns. In some embodiments, the first opening 106 and/or the second opening 108 of the microbubbles 104 have a width or diameter of about 5 microns, 10 microns, 20 microns, 30 microns, 40 microns, 50 microns, 60 microns, 70 microns, 80 microns, 90 microns, 100 microns, about 150 microns, about 200 microns, about 250 microns, about 300 microns, about 350 microns, about 400 microns, about 450 microns, about 500 microns, about 550 microns, about 600 microns, about 650 microns, about 700 microns, about 750 microns, about 800 microns, about 850 microns, about 900 microns, about 950 microns, or about 1000 microns. In some embodiments, the first opening 106 and/or second opening 108 of the microbubbles 104 have a width or diameter ranging between 5 microns and 1000 microns, between 10 microns and 950 microns, between 15 microns and 900 microns, between 20 microns and 800 microns, between 25 microns and 700 microns, between 30 microns and 600 microns, between 35 microns and 500 microns, between 40 microns and 450 microns, between 45 microns and 400 microns, between 50 microns and 350 microns, between 55 microns and 300 microns, between 60 microns and 250 microns, between microns 65 and 225 microns, between 70 microns and 200 microns, between 75 microns and 175 microns, between 80 microns and 150 microns, between 85 microns and 125 microns, between 90 microns and 120 microns, or between 95 microns and 110 microns. In some embodiments, the first opening 106 may have a different width or diameter than the second opening 108.

In some embodiments, the first opening 106 has a larger width or diameter than the second opening 108. In some embodiments, the first opening 106 has a width or diameter 1.5×, 2×, 3×, 4×, 5×, 6×, 7×, 8×, 9×, or 10× the width or diameter of the second opening 108. In some embodiments, the first opening 106 has a smaller width or diameter than the second opening 108. In some embodiments, the width or diameter of the first and second openings may be the same. In some embodiments, the plurality of microbubbles 104 may have an aspect ratio (microbubble diameter/microbubble openings) of about 1, about 1.5, about 2, about 2.5, about 3, about 3.5, about 4, about 4.5, about 5, or in the range between 1 and 5. In some embodiments, the aspect ratio may be about 2.5, providing optimal nutrient and waste exchange, preventing lactate build-up, and allowing for the concentration of cell secreted factors that can positively affect the biology.

In some embodiments, each microbubble of the plurality of microbubbles 104 may have an inner volume of about 1 nL, about 5 nL, about 10 nL, about 20 nL, about 30 nL, about 40 nL, about 50 nL, about 60 nL, about 70 nL, about 80-nL, about 90 nL, about 100 nL, about 500 nL, about 1 μL, about 2.5 μL, about 5 μL, about 7.5 μL, about 10 μL, or in the range between 5 nL and 10 μL. In some embodiments, the MB membrane 102 may have a thickness of about 0.01 mm, about 0.05 mm, about 0.1 mm, about 0.5 mm, about 1 mm, about 1.2 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 3 mm, about 5 mm, or in the range between 0.01 mm and 5 mm.

In some embodiments, the MB membrane 102 comprises or is formed from any material known to one of skill in the art. Examples include but are not limited to glass, paper, hydrogels, silicon, metals, and polymers (e.g. poly-L-lysine, acrylics, elastomers, polydimethylsiloxane (PDMS), polymethylmethacrylate (PMMA), polyimide, SU-8, parylene, liquid crystal polymers (LCPs), polyacrylamide, polystyrene, polymers with soluble gas, or various functionalized polymers such as those with amine, epoxy, carboxyl, or azide groups). In some embodiments, the MB membrane 102 comprises or is formed from PDMS. In some embodiments, the MB membrane 102 comprises or is formed from soft PDMS or stiff PDMS. In some embodiments, the MB membrane 102 may comprise any clear or optically transparent material known to one of skill in the art, allowing for in situ imaging of cells in the MB array.

In some embodiments, the MB array 100 and/or MB membrane 102 comprises a multi-well array, for example a round well array, a square well array, a triangular well array, a hexagonal well array, a flat-bottom well array, a V-bottom well array, a U-bottom well array, a C-bottom well array, an F-bottom well array. In some embodiments, the MB membrane 102 may comprise any well array as would be known to those skilled in the art. In some embodiments, the MB membrane 102 may comprise any membrane or substrate having a plurality of cavities or wells having any shape or size.

In some embodiments, the nanoporous membrane 110 comprises a top side or surface, a bottom side or surface and further comprises pores having a diameter between 1 nm and 10,000 nm. In some embodiments, the thickness of the nanoporous membrane 110 ranges between 50 nm and 500 nm. In some embodiments, the nanoporous membrane 110 comprises or is formed from silicon (e.g. silicon nitride). In some embodiments, the nanoporous membrane 110 contains an ultrathin (≈100 nm thick) optically clear, porous silicon nitride membrane patterned in a window (e.g., a 700 μm×2 mm window). In some embodiments, the window has slots or openings (e.g., slots 118 described herein). In some embodiments, the nanoporous membrane 110 features dual-scale porosity with 5 μm pores superimposed on a nanoporous silicon nitride background. The 5 μm pores provide portals for cell transmigration, while the nanopores (≈60 nm diameter; ≈15% porosity) allow unhindered autocrine/paracrine signaling by small molecule exchange throughout the membrane interface between the fluid paths or components. In some embodiments, the nanoporous membrane 110 separates at least the first and second cell types (112, 114) while allowing for communication between the cell types on either side of the membrane, allowing them to form physiologically relevant 3D tissue barriers. Furthermore, the transmigration of small molecules may occur across nanoporous membrane 110. Additionally, advantages of nanoporous membrane 110 include resistance free transport of soluble bioactive factors between the cell types as well, direct cell-to-cell contact that can occur via extension of cell dendrites through the pores in the membrane, and optically clear imaging of cells positioned within the MB array 100.

In some embodiments, the nanoporous membrane 110 may be fluidly connected to any number of fluid channels, pumps, and/or sensors, and may further comprise any number of supporting structures or support layers. In some embodiments, the nanoporous membrane 110 allows for resistance-free transport of soluble bioactive factors between the plurality of microbubbles 104 and the bottom side of the nanoporous membrane 110. In some embodiments, the MB membrane 102 may be coupled to the nanoporous membrane 110 using oxygen plasma treatment, or any adhesive known by one of ordinary level of skill in the art, such as pressure sensitive adhesives (PSA). In some embodiments, MB array 100 comprising the coupled MB membrane 102 and the nanoporous membrane 110 may be referred to herein in some examples as a “hybrid platform.”

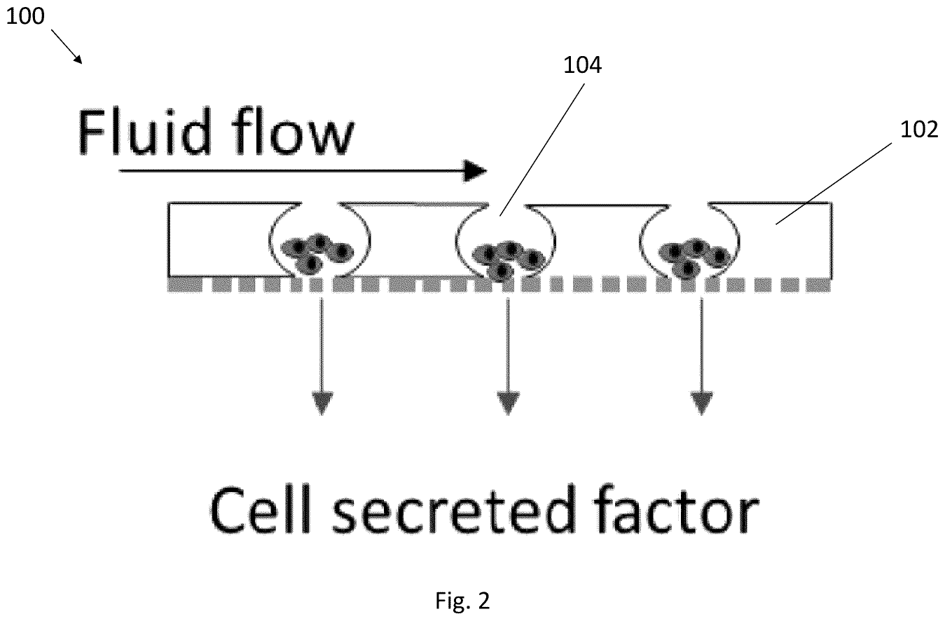

In some embodiments, the MB array 100 may further comprise at least one top channel or fluid path positioned above MB membrane 102 and fluidly connected to interior volumes of at least a portion of the plurality of microbubbles 104 via the first openings 106. In some embodiments, the at least one top channel or fluid path may further be fluidly connected to any number of fluid or solution reservoirs, pumps, valves, or the like, and any combinations thereof. In some embodiments, fluid flow within the top channel may be used to control fluid flow fields that enter at least a portion of the microbubbles to introduce mixing and shear stress within the plurality of microbubbles 104. In some embodiments, the top channel may be configured to provide a fluid path for a fluid across the top surface of the MB array 100. In some embodiments, the top channel may be configured to provide nutrients to cells cultured or positioned in the plurality of microbubbles 104. In some embodiments, the top channel may be configured for waste removal from the plurality of microbubbles 104. In some embodiments, the fluid may comprise cells, cell culture media, buffers, stimulants, biomolecules, drugs or drug candidates, therapeutics or therapeutic candidates, water, blood, or any combinations thereof. FIG. 2 depicts the fluid flow from the top channel or fluid path above the MB membrane 102.

In some embodiments, any of the cell types for MB array 100 may be chosen to replicate a salivary gland. In some embodiments, any of the cell types for MB array 100 (e.g., the first cell type 112 and/or the second cell type 114) may be chosen from the group consisting of: acinar cells (e.g. seromucous cells, serous cells) intercalated duct cells (e.g. Gstt1+/Smgc+, Gfra3+/Kit+, K5+/K14+/Axin2+), myoepithelial cells, non-epithelial cells (e.g. fibroblasts, macrophages, natural killer (NK) cells, dendritic cells, T cells), duct cells (e.g. granular convoluted tubule (GCT) cells, basal cells, ionocytes, striated/excretory cells), vascular cells, nerve cells, endothelial cells, immune cells, and SG tissue cells. In some embodiments, the first cell type 112 may comprise SG cells, SG organoids, SG cell clusters, SG tissue cells, and/or oral cells. In some embodiments, the second cell type 114 may comprise endothelial cells, nerve cells, vascular cells, and/or immune cells.

In some embodiments, any of the cell types for MB array 100 may be chosen to replicate a retina. In some embodiments, any of the cell types for MB array 100 (e.g., the first cell type 112 and/or the second cell type 114) may be chosen from the group consisting of: retinal cells, retinal cell clusters, retinal organoids, retinal pigment epithelium (RPE) cells, neural cells, non-neuronal cells (e.g. Muller cells), and vascular cells.

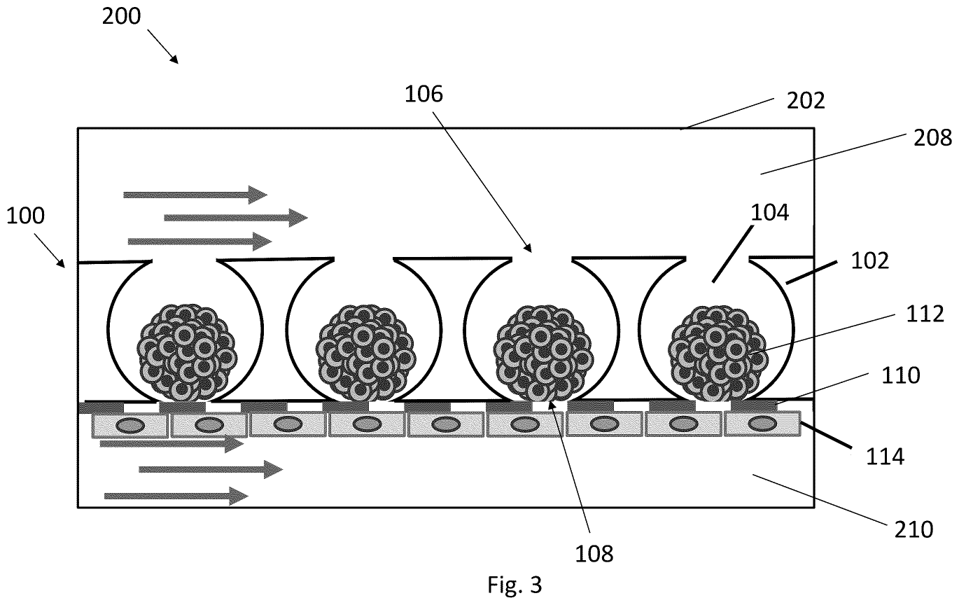

In some embodiments, any of the cell types for MB array 100 may be chosen to replicate a tumor. In some embodiments, any of the cell types for MB array 100 (e.g., the first cell type 112 and/or the second cell type 114) may be any cancer cell culture including but not limited to squamous cell carcinoma (SCC) cells, melanoma cells (e.g. A375P, A375MA1, A375MA2) tumor cells, tumor cell clusters, or tumor organoids. (Pu, Qihui et al. “Identifying drug resistant cancer cells using microbubble well arrays.” Biomedical microdevices vol. 19,3 (2017): 17. doi:10.1007/s10544-017-0160-9; Chandrasekaran, Siddarth et al. “In vitro assays for determining the metastatic potential of melanoma cell lines with characterized in vivo invasiveness.” Biomedical microdevices vol. 18,5 (2016): 89. doi:10.1007/s10544-016-0104-9) Aspects of the present invention relate to exemplary microfluidic devices comprising at least one disclosed MB array 100. Referring now to FIG. 3, shown is an exemplary microfluidic device 200. Generally, microfluidic device 200 comprises a housing 202 at least partially enclosing at least one MB array 100 and forming fluid paths to and from portions of the MB array 100. In some embodiments, the nanoporous membrane 110 allows for resistance-free transport of soluble bioactive factors between, or to at least a portion of plurality of microbubbles 104 and/or MB membrane 102, or between the plurality of microbubbles 104 and the bottom side or surface of the nanoporous membrane 110.

In some embodiments, the microfluidic device 200 may further comprise at least one top fluid path or top channel 208 in the housing 202 positioned above MB array 100 and/or MB membrane 102 and fluidly connected to the interior volumes of at least a portion of the plurality of microbubbles 104 via the top openings 106. In some embodiments, the microfluidic device 200 may further comprise at least one bottom fluid path or bottom channel 210 in the housing 202 positioned below the nanoporous membrane 110 and fluidly connected to the nanoporous membrane 110, and to bottom openings 108 of MB membrane 102 through the pores of nanoporous membrane 110.

In some embodiments, the microfluidic device 200 allows for the integration of one or more organ components or cell cultures by comprising a plurality of MB arrays 100. In some embodiments, the first cell type 112 and the second cell type 114 are cultured to form a vascularized, innervated network of cells, tissues, or organs. In some embodiments, the first cell type 112 and the second cell type 114 are chosen to replicate SG microstructure and/or tissue. In some embodiments, the first cell type 112 and second cell type 114 may be the same cell type, or different cell types. In some embodiments, the device 200 may comprise a plurality of MB arrays 100 comprising a third cell type, a fourth cell type, or a fifth cell type. In some embodiments, the device 200 may comprise any number of MB arrays 100 and any number of cell types, forming complex tissue microstructures, organs and/or recapitulating one or more physiological systems. In some embodiments, device 200 may comprise a plurality of MB arrays 100 stacked on top of one another or placed side-by-side on any of the disclosed devices connected by any number of fluid paths, channels and/or conduits.

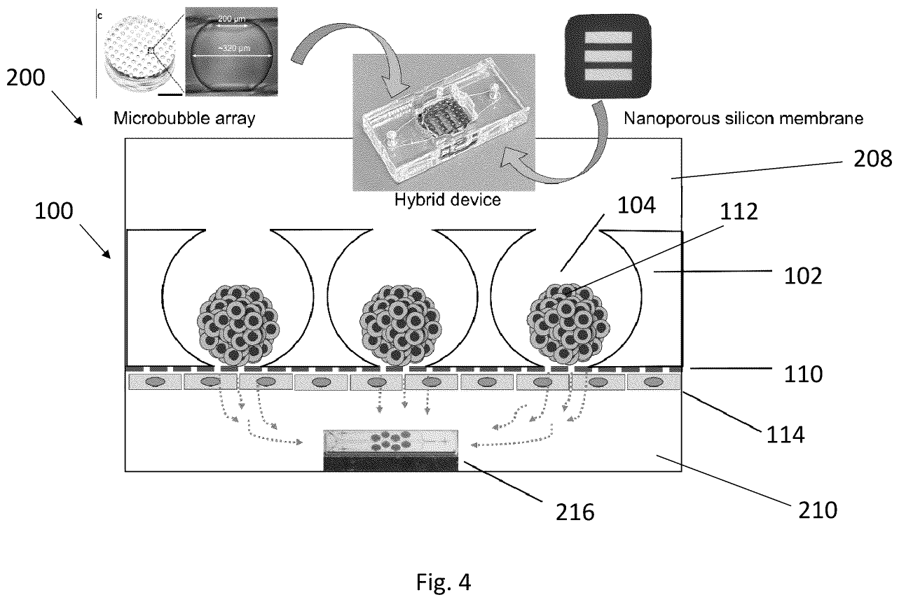

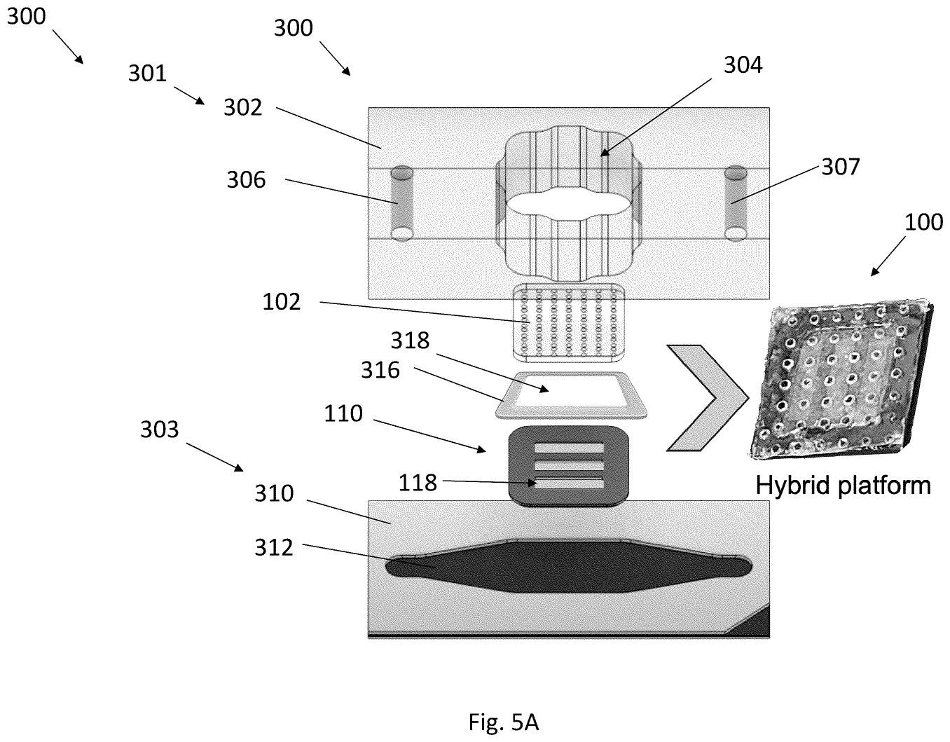

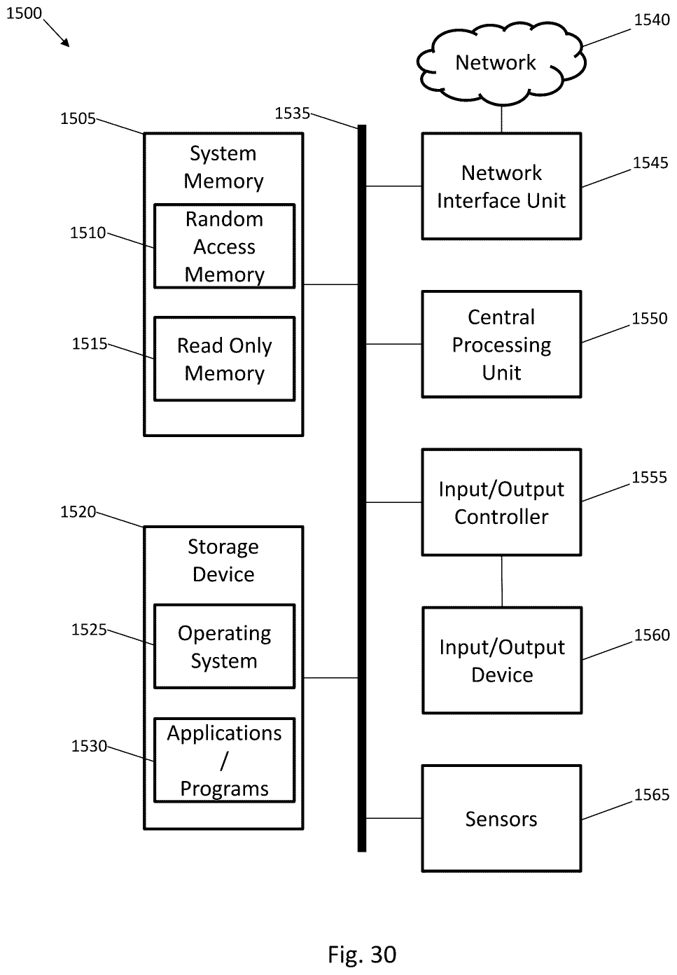

Referring now to FIG. 4, in some embodiments the microfluidic device 200 further comprises a sensor 216 positioned below the nanoporous membrane 110 within the bottom channel 210. In some embodiments, the sensor 216 may be an optical sensor or photonic array, configured to assess cell secreted factors or to optically interrogate each microbubble independently. In some embodiments, the microfluidic device 200 may further comprise a resistance sensor coupled to the MB membrane 102, configured to measure the electrical resistance of the MB membrane 102. In some embodiments, the microfluidic device 200 may further comprise any number of biosensors, flow rate sensors, fluid pressure sensors, temperature sensors, pH sensors, oxygen sensors, carbon dioxide sensors, electrochemical sensors, or the like, and any combinations thereof. In some embodiments, the sensors of the device 200 may be communicatively connected to a computing device (for e.g. computer 1500 described herein and depicted in FIG. 30).

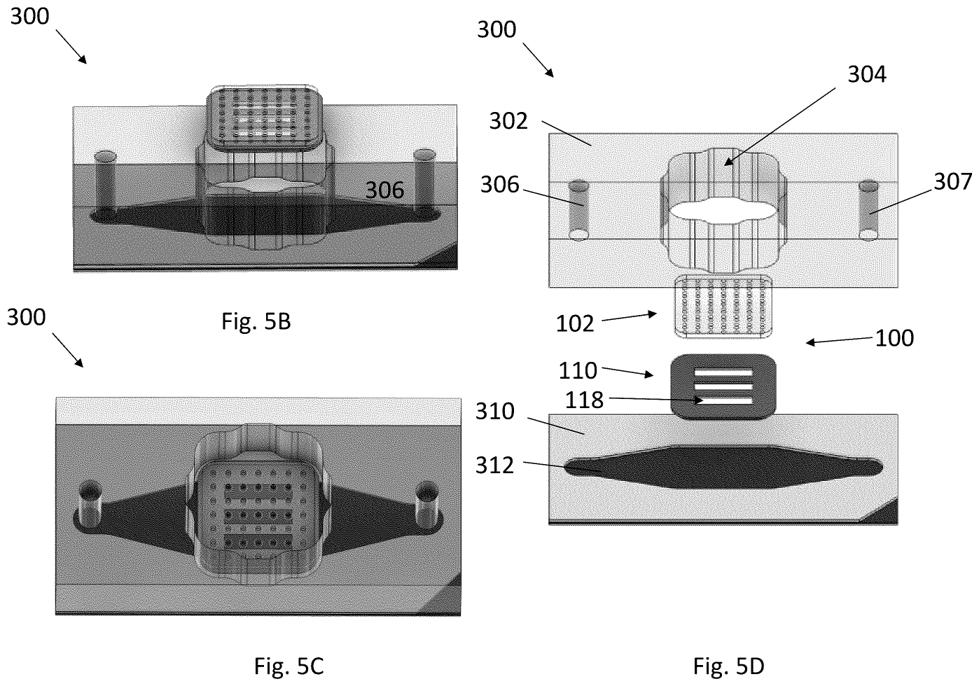

Aspects of the present invention relate to a microphysiological system 300 comprising at least one MB array 100. Referring now to FIG. 5A, shown is an exemplary microphysiological system 300. Generally system 300 comprises a top portion 301 having a body 302 with at least one cavity 304, an inlet 306 and an outlet 307, each passing through the body 302, at least one MB array 100 (the MB array 100 comprising a MB membrane 102 bonded to a nanoporous membrane 110) positioned within the at least one cavity 304, and a bottom portion 303 comprising a plate 310 having an indentation forming a channel 312, wherein the top portion 301 is fixedly and removably attached to the bottom portion 303 and the cavity 304, inlet 306, and outlet 307 are fluidly connected to, and by, the channel 312. In some embodiments, the nanoporous membrane 110 comprises one or more slots 118. In some embodiments, the nanoporous membrane 110 may comprise one or more slots 118 or grooves, either forming indentations in, or passing entirely through the membrane. In some embodiments, MB array 100 is configured such that the plurality of microbubbles 104 of MB membrane 102 are positioned above or aligned with the one or more slots 118 of the nanoporous membrane 110. In some embodiments, the pattern of the plurality of microbubbles 104 are aligned at an angle with respect to slots 118. In some embodiments, the pressure sensitive adhesive 316 may comprise one or more openings corresponding the one or more slots 118 in the membrane 110. In some embodiments, the nanoporous membrane 110 comprises a non-membrane layer (e.g., a window) with one or more slots 118 with the nanoporous membrane positioned or layered on the non-membrane layer. FIGS. 5B-5D depict additional views of the system 300.

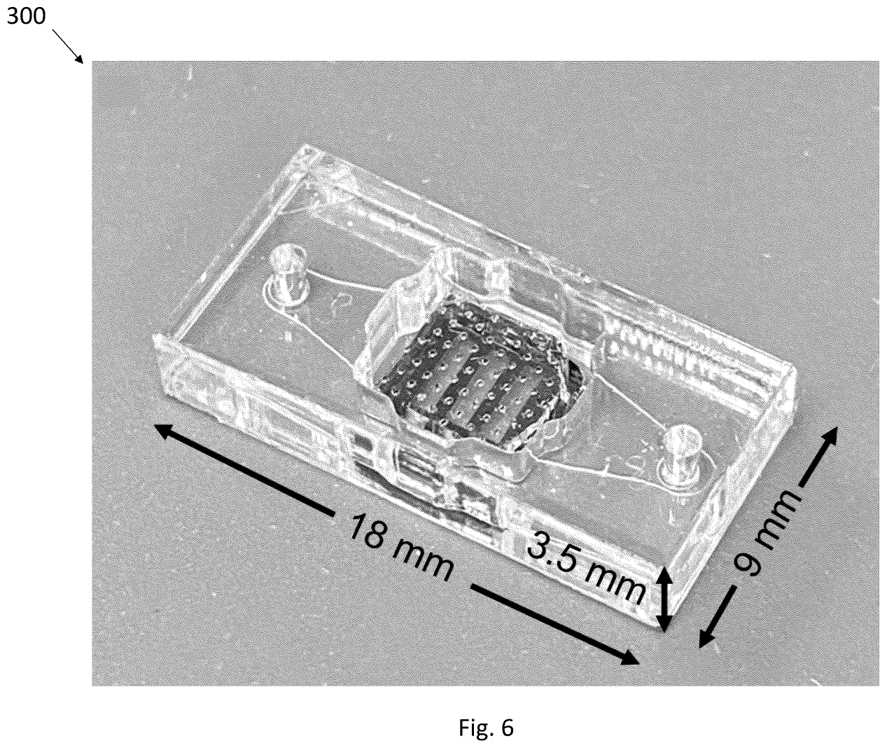

In some embodiments, the body 302 may have a generally rectangular shape. In some embodiments, the cavity 304 may have a spherical shape, an ovular shape, a rectangular shape, a square shape or an irregular shape. In some embodiments, the cavity 304 may comprise ridged edges. In some embodiments, the body 302 may comprise any material known to one of skill in the art. For example, metals, metal alloys, plastics, organic or non-organic polymers may be used. In some embodiments, and referring now to FIG. 6, the body 302 may have a length ranging between 5 mm and 200 mm, a width ranging between 2 mm and 100 mm, and a thickness ranging between 1 mm and 50 mm.

In some embodiments, the nanoporous membrane 110 has a top surface and a bottom surface and is positioned within the channel 312 and beneath the MB membrane 102. In some embodiments, the nanoporous membrane 110 is coupled to the MB membrane 102 via a spacing portion, which in some embodiments is a pressure sensitive adhesive 316 with a central opening 318. In some embodiments, the pressure sensitive adhesive 316 may be cut to form one or more central openings. In some embodiments, the spacing portion forms a gap (e.g., gap 1111) with closed volume between the bottom surface of the MB membrane 102 and the top surface of the nanoporous membrane 110. The gap may be filled with natural or synthetic polymers and/or hydrogels engineered to support tissue and cell growth. Filling the gap with a polymer or hydrogel creates a base to the plurality of microbubbles 104 for cell seeding. In some embodiments, the MB membrane 102 may be coupled to the nanoporous membrane 110 such that no gap is formed.

In some embodiments, the gap may have a height ranging between 0 μm and 500 μm. In some embodiments, the gap forms a volume ranging between 0 μL and 10 μL. In some embodiments, the gap may comprise a fluid. In some embodiments, the gap may comprise a hydrogel. In some embodiments, the hydrogel comprises one or more polymers, including but not limited to, polyethylene glycol (PEG), Matrigel, collagen, fibrinogen, alginate, polyacrylamide, hyaluronic acid, and the like. In some embodiments, one or more cell types are embedded or encapsulated within the hydrogel. In some embodiments, the hydrogel can be cross-linked, such as by photo-cross-linking, thermal cross-linking, chemical cross-linking, and the like. In some embodiments, the top or bottom surface of the nanoporous membrane 110 may be treated with a surface treatment. Surface treatments may be used to prevent or enhance the interaction of cell/tissue seeded in the plurality of microbubbles 104 with those seeded in the gap/gel. In some embodiments, the surface treatment may be Pluronic F-127, polyvinyl alcohol (PVA), silanes, antibodies, albumin, and other proteins, peptides, oligonucleotides, or chemical coupling agents as would be known to those skilled in the art.

In some embodiments, a fluid flow may be established into and out of the bottom channel 312 through the inlet 306 and outlet 307. In some embodiments, the inlet 306 and outlet 307 are openings or channels forming lumens extending from a top surface of the body 302 to a bottom surface of the body 302. In some embodiments, the inlet 306 and outlet 307 may be formed by one or more fluid paths, conduits, or channels. In some embodiments, the inlet 306 and outlet 307 may be fluidly connected to external conduits, pumps, fluid or solution reservoirs, or the like, and any combinations thereof. In some embodiments, the inlet 306 and outlet 307 may be fluidly connected to the cavity 304 via additional channels within the body 302 and be configured to establish a fluid path above MB membrane 102. In some embodiments, the fluid may be a cell culture media and/or other solutions as would be known to one of skill in the art. For example, the fluid may comprise cells, cell culture media, buffers, stimulants, biomolecules, drugs or drug candidates, therapeutics or therapeutic candidates, water, blood, or any combinations or solutions thereof. In some embodiments, the fluid may have a flow rate ranging between 0 μL/min and 2000 μL/min. In some embodiments, the fluid has a preferred flow rate ranging between 5 μL/min and 100 μL/min. As known to those skilled in the art, the flow rates may be adjusted to control shear stress depending on the system design of micro- and milli-fluidic devices.

In some embodiments, the system 300 may further comprise one or more sensors configured to measure or interrogate aspects of the MB array 100. In some embodiments, the one or more sensors are positioned within channel 312 or below the nanoporous membrane 110. The one or more sensors may comprise optical sensors, photonic arrays, biosensors, flow rate sensors, fluid pressure sensors, temperature sensors, pH sensors, oxygen sensors, carbon dioxide sensors, electrochemical sensors, transelectrical resistance (TEER) sensors, and the like. In some embodiments, the one or more sensors may be configured to assess cell secreted factors or optically interrogate each microbubble of plurality of microbubbles 104 independently. In some embodiments, the one or more sensors may be configured to measure the electrical resistance of the MB membrane 102. In some embodiments, the one or more sensors may monitor fluid flow velocity or pressure in and out of the system 300. In some embodiments, the one or more sensors may return measurements to a computer and/or interface device (for e.g. computer 1500 of FIG. 30).

In some embodiments, the body 302 may comprise one or more cavities 304, each cavity at least partially enclosing and holding an MB array 100. In some embodiments, the plate 310 may comprise one or more channels 312. In some embodiments, the MB array 100 at least partially resides within cavity 304 and the one or more channels 312. In some embodiments, the MB array 100 is at least partially positioned within the one or more channels 312. In some embodiments, the one or more cavities 304 may be interconnected via channels, conduits, inlets, or outlets. In some embodiments, the system 300 may be fluidly connected to one or more systems 300.

In some embodiments, the microphysiological system 300 allows for the integration of one or more organoids or cell cultures, wherein at least a first and a second cell type (112, 114) may be disposed or cultured within the system 300. In some embodiments, the organoids or cell cultures may be configured to replicate the SG microstructure or microenvironment. In some embodiments a first cell type 112 is disposed or cultured within MB membrane 102, and a second cell type 114 is disposed or cultured on the bottom surface of the nanoporous membrane 110. In some embodiments, a third cell type is disposed or cultured in the gap 111 between the MB membrane 102 and the nanoporous membrane 110, wherein the third cell type is embedded or encapsulated in a hydrogel within the gap 111. In some embodiments, the microphysiological system 300 further comprises a fourth cell type or a fifth cell type. In some embodiments, MB array 100 may comprise a plurality of cell types positioned in patterns or arrangements across the MB membrane 102 and/or nanoporous membrane 110.

In some aspects, the present invention relates to a method of screening one or more drug/therapeutic candidates for their effect on SGs, or any other recapitulated tissues or organs discussed herein. Generally, the method of drug screening comprises the steps of providing any of the MB arrays, membranes, or devices and systems disclosed herein, seeding a first cell type in at least a portion of the plurality of microbubbles 104, contacting and/or flowing a test agent or drug/therapeutic candidate across the cells, and monitoring a response of the test agent or drug/therapeutic candidate.

In some embodiments, the method of drug screening may further comprise seeding a second cell type on a bottom surface of the nanoporous membrane 110. In some embodiments, the method may further comprise introducing a hydrogel comprising a third cell type in the gap 111 between the MB membrane 102 and the nanoporous membrane 110. In some embodiments, the method may further comprise flowing a cell culture media, buffer, stimulant, blood, or solution across the cells. In some embodiments, the method may further comprise manipulating one or more conditions to replicate an SG microenvironment. In some embodiments, the one or more conditions may include temperature, light, electrical stimulation, fluid pressure, fluid flow rate, fluid flow shear force, and the like, or any combinations thereof.

In some embodiments, the step of monitoring a response of the test agent or drug/therapeutic candidate may comprise optically interrogating cells in each microbubble of at least a portion of the plurality of microbubbles 104 via fluorescent or bright field microscopic imaging, RT-PCR analysis, calcium flux analysis, use of an optical sensor or photonic array, collecting and assessing cell secreted factors from the plurality of microbubbles 104, measuring a resistance of cells or tissue via a resistance sensor, monitoring pH levels, oxygen levels, carbon dioxide levels, fluid pressure, or fluid flow rates, or harvesting the first, second or third cell types for analysis (e.g. via immunohistochemistry, cellular composition analysis, flow cytometry, RNA sequencing, and other measures of cell and tissue function and analysis known to those skilled in the art).

In some aspects, the MB array 100, the microfluidic device 200 or the microphysiological system 300 described above may be utilized for disease recapitulation and enable high-throughput and high-content drug screening and toxicology testing with more reliable drug discovery results. In some embodiments, the MB array 100, the device 200 and/or the system 300 enable spatial-temporal control of microenvironments and mechanical cues modeling. In some embodiments, the MB array 100, the device 200 and/or the system 300 enable multi-tissue or multi-organ interaction, better mimicking the features of in vivo natural systems. In some embodiments, the MB array 100, the device 200 and/or the system 300 enable the integration of biosensing and bioimaging sensors or equipment. In some embodiments, the MB array 100, the device 200 and/or the system 300 are capable of reducing the need for animal use in drug development.

In some embodiments, the disclosed technology provides an innervated and vascularized SG organ-on-a-chip model for advanced disease modeling, drug screening, and toxicology testing. Any disclosed device or system may be connected or coupled one or more microphysiological devices, such as a multi-organ-on-a chip device, or a dental microphysiological device. In some embodiments, the disclosed device or system comprises an SG chip coupled to a dental microphysiological system. In some embodiments, an output of any disclosed device or system is directed to one or more microphysiological device, such as a dental microphysiological system or dental tissue microphysiological system.

In some embodiments, the MB array 100 and/or MB membrane 102 of the present application may be integrated with any known tissue-on a chip devices, organ-on-a-chip devices, multi-organ-on-a-chip devices, or tumor-on-a-chip devices. In some embodiments, the MB array 100 and/or MB membrane 102 of the present application, may be used for replication of tissues or organs such as eyes, retinas, salivary glands, dental tissue, lungs, heart, skeletal muscles, gastrointestinal (GI) tract kidneys, or stomach. In some embodiments, the MB array 100 of the present invention may be used to replicate disease states or conditions such as dental cavities, tumors, eye diseases, lung diseases, kidney diseases, or stomach diseases. In some embodiments, the MB array 100 or any of the devices comprising the MB array 100 described herein may be coupled to a tooth chip for discovery of drugs that prevent pathogenic bacteria that cause plaque and cavities.

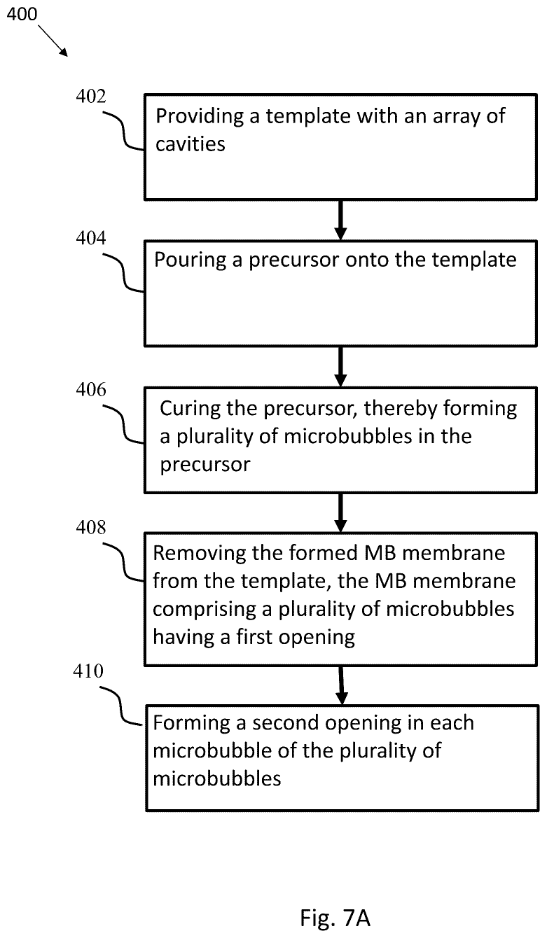

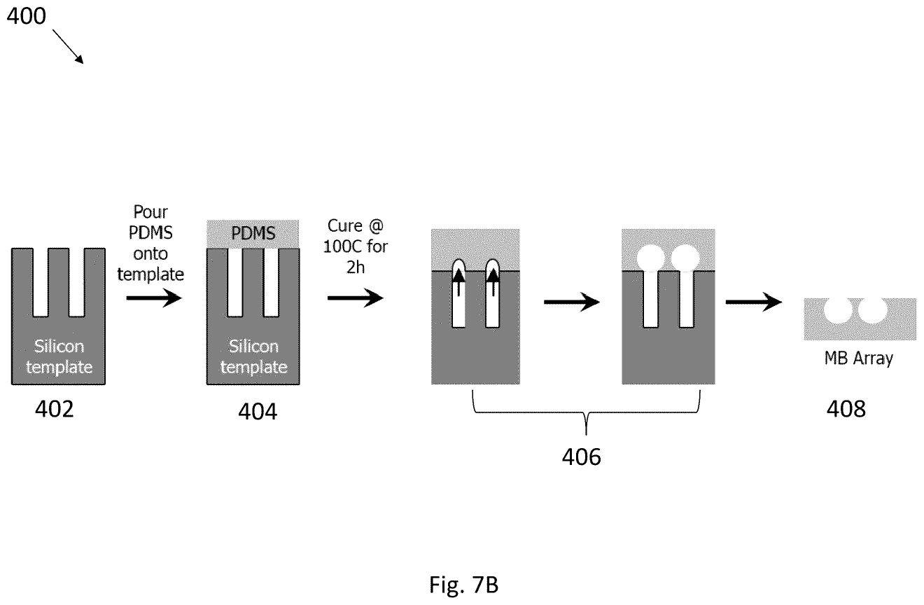

In some aspects, the present invention relates to methods of forming an MB membrane or array. Referring now to FIG. 7A, described is an exemplary method 400 of manufacturing or forming an MB membrane or array. The method 400 generally comprises providing a template comprising an array of cavities or pits (402), pouring a precursor (e.g., PDMS) onto the template (404), curing the precursor (e.g., at 100° C. for 2 h) thereby forming a plurality of microbubbles in the precursor (406), removing the formed MB membrane or array from the template, the MB membrane or array comprising one or more microbubbles each having a first opening (408), and forming a second opening in each microbubble of the plurality of microbubbles (410). FIG. 7B depicts an illustrative schematic of the method 400.

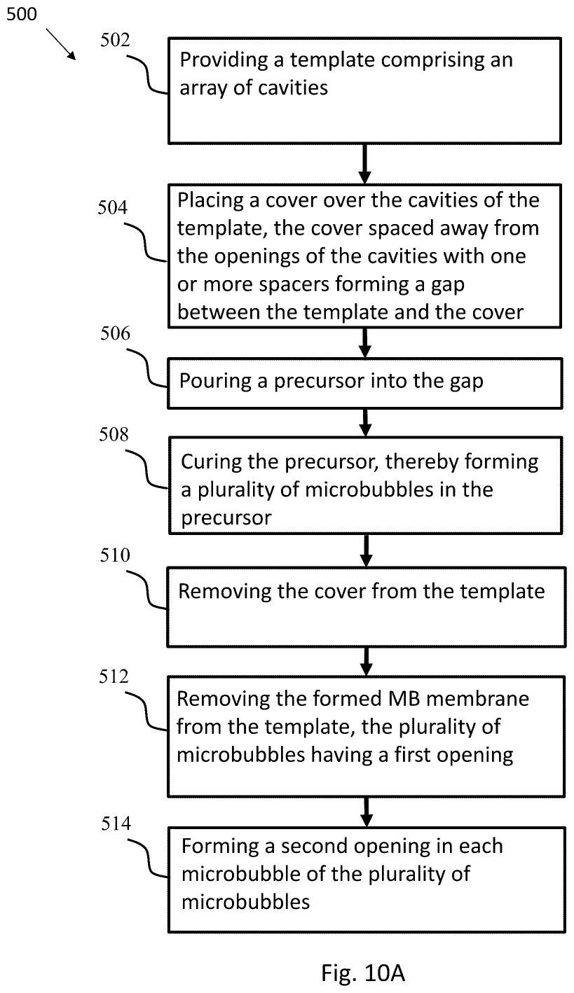

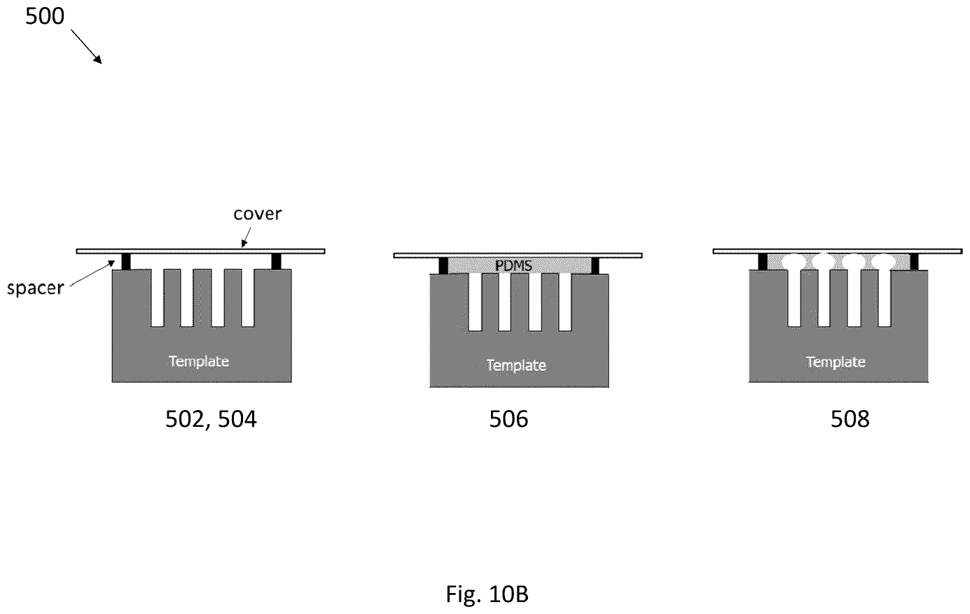

Referring now to FIG. 10A, shown is a flowchart of an exemplary restricted gas expansion molding (rGEM) molding method 500 of forming a double-sided open MB membrane. In some embodiments, method 500 comprises the steps of: providing a template comprising an array of cavities or pits (502), placing a cover over the cavities or pits of the template, the cover spaced away from the openings of the cavities with one or more spacers forming a gap between the template and the cover (504), pouring a precursor into the gap (506), curing the precursor, thereby forming a plurality of microbubbles in the precursor (508), removing the cover from the template (510), removing the formed microbubble membrane from the template, the plurality of microbubbles having a first opening (512), and forming a second opening in each microbubble of the plurality of microbubbles (514). FIG. 10B depicts an illustrative schematic of the method 500. The spacer height is configured to restrict the growth of the microbubble that forms over the cavities. In some embodiments, when the precursor is cured, the microbubble is formed, with its growth or expansion restricted by the height of the spacer and the cover. It should be appreciated that the height of the one or more spacers may be modified to create microbubbles of any desired size/diameter.

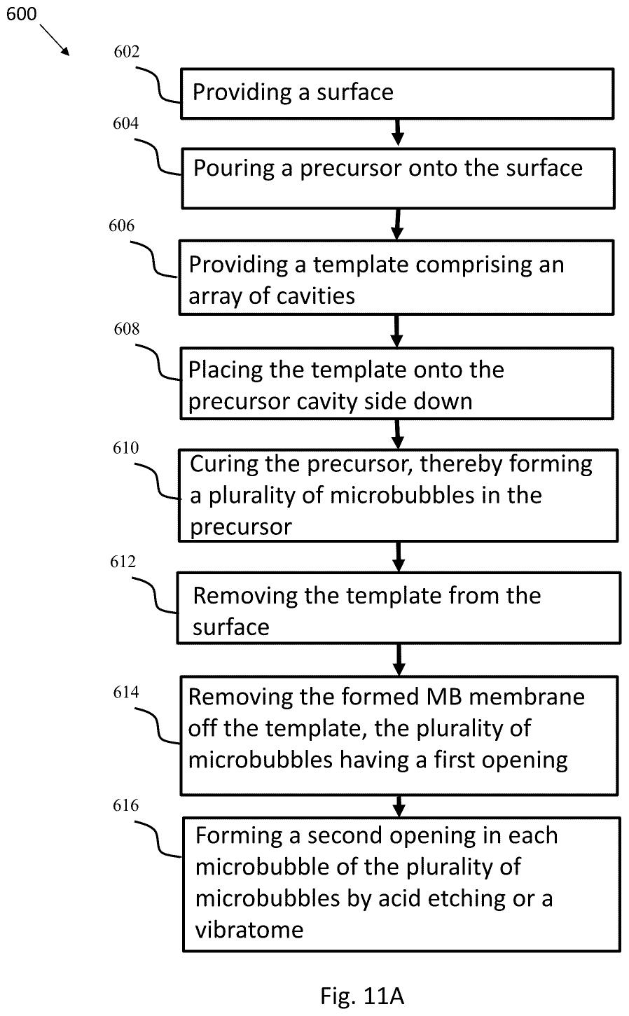

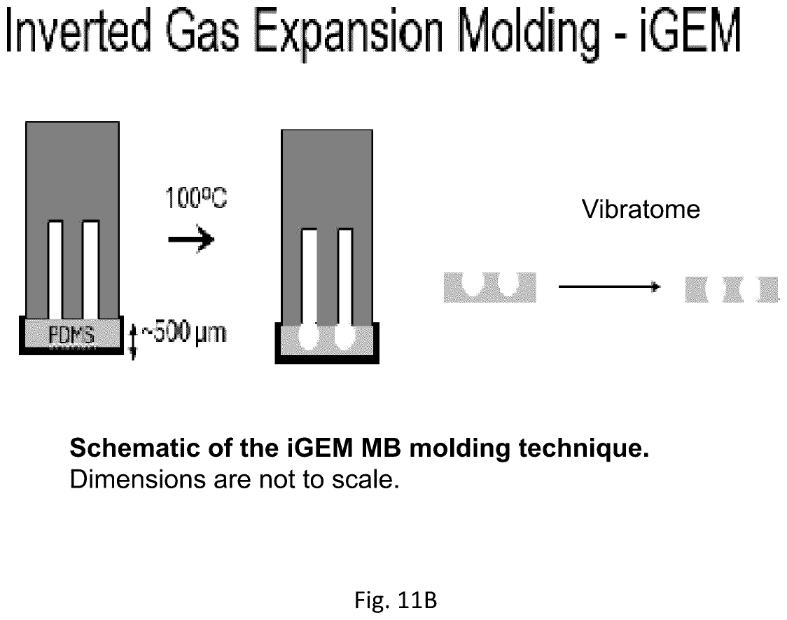

Referring now to FIG. 11A, shown is a flowchart illustrating an exemplary inverted gas expansion molding (iGEM) method 600 of forming a double-sided open microbubble membrane. The method 600 may generally comprise the steps of: providing a surface (602), pouring a precursor onto the surface (604), providing a template comprising an array of cavities or pits (606), placing the template onto the precursor cavity or pit side down (608), curing the precursor thereby forming a plurality of microbubbles in the precursor (610), removing the template from the surface (612), removing the formed microbubble membrane off the template, the plurality of microbubbles having a first opening (614), and forming a second opening in each microbubble of the plurality of microbubbles by acid etching or using a vibratome (616). In some embodiments, the first and second openings of each microbubble of the plurality of microbubbles are positioned diametrically opposite each other. In some embodiments, the method 600 may 27 tilizeed when a particular thickness for the MB membrane is desired. FIG. 11B depicts an illustrative schematic of the method 600.

In some embodiments, the first and second openings of each microbubble of the plurality of microbubbles are formed diametrically opposite each other. In some embodiments, the precursor is cured for 100° C. for 2 h. In some embodiments, the template may comprise silicon, polymers (such as PDMS, photoresists) or non-organic materials (such as glass, nitrites, metals, metal alloys, and the like). In some embodiments, the precursor comprises PDMS, mixtures of soft and rigid PDMS or other elastomeric polymers or polymers with dissolved gas. In some embodiments, the cover may comprise materials including, but not limited to, glass, silicon, or Teflon. In some embodiments, a thin precursor film may form during microbubble expansion but may be removed when the cover is lifted off. In some embodiments, the cover may be treated using agents to increase or decrease binding to the expanding polymer that may form a thin precursor film to ensure that that a top opening forms when the cover is removed. For example, trialkoxysilanes functionalized with hydrophobic groups to repel or alkenes that bind the expanding polymer may be used. In some embodiments, the MB membrane or array may be formed via any known 3D printing technologies or other methods such as ink jet printing, laser printing, or UVR forming.

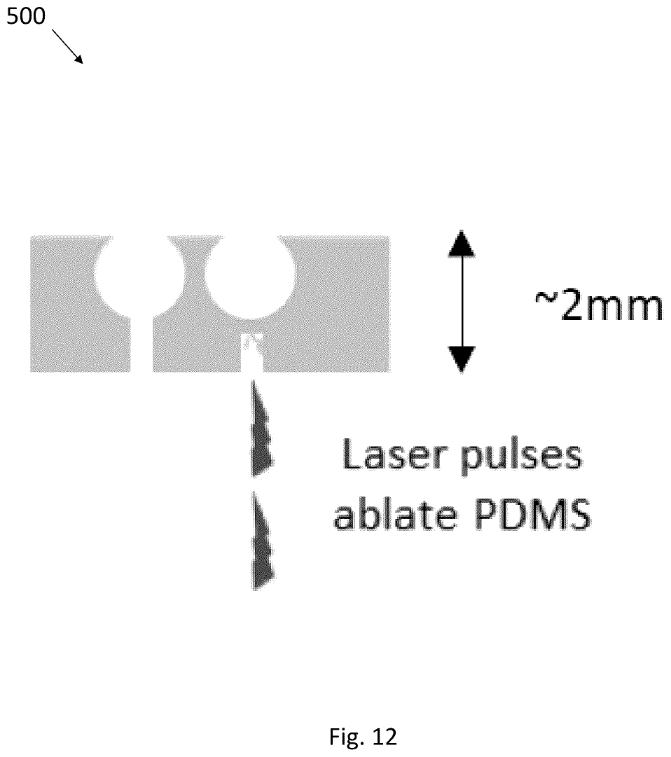

In some embodiments, the step of forming the second openings of each microbubble of the plurality of microbubbles in the microbubble membrane is performed via inserting the single opening MB membrane or array, with or without the template into a vibratome. In some embodiments, forming the second openings of each microbubble of the plurality of microbubbles may comprise laser ablation, chemical etching, or reactive ion gas phase etching to form the second opening. In some embodiments, the laser ablation step comprises utilizing laser pulses to ablate the precursor on the bottom surface of the MB membrane or array to create bottom openings of the one or more microbubbles (as depicted in FIG. 12). In some embodiments, the chemical etching step comprises immersing at least a portion of the membrane in a sulfuric acid 98% solution or a sulfuric acid solution mixed with hydrogen peroxide until the bottom openings are created. In some embodiments, the reactive ion gas phase etching step comprises optimizing the combination of O₂, SF₆, and CHF₃ to chemically form the bottom openings.

In some embodiments, the template comprises between 1 and 2000 wells, each well forming one microbubble in the produced MB array. In some embodiments, the one or more spacers have a height of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, or in a range between 0.1 mm and 2.5 mm.

In some aspects, the present invention relates to a method of seeding a microbubble array, comprising the steps of providing an MB array, introducing or disposing a first cell type culture into at least a portion of the microbubbles of the plurality of microbubbles and introducing or disposing a second cell type culture below the porous nanomembrane. In some embodiments, and prior to the step of introducing or disposing the first cell type culture, the method further may comprise the steps of: providing a solution comprising a third cell type mixed with a hydrogel and crosslinking agent, flowing the solution through at least a portion of the microbubbles of the microbubble membrane into a gap between the MB membrane and the nanoporous membrane, and polymerizing the solution such that the third cell type is embedded within a cross-linked hydrogel in the gap between the MB membrane and the nanoporous membrane. In some embodiments, the crosslinking agent is UV activated. In some embodiments, the gel may be formed with temperature or mixing reagents at room temperature. In some embodiments, the method further comprises a step of establishing a flow of fluid across the MB array or in the bottom channel. In some embodiments, the fluid may be a cell culture media with and without drugs or cell stimulants, imaging reagents, or other chemical and biological reagents as known to those skilled in the art.

Aspects of any disclosed device or system may be electronically and/or communicatively connected to a computing device (e.g., computer 1500 depicted in FIG. 30). This includes any microfluidic devices, microphysiological systems and any pumps, valves, sensors, thereof. In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

FIG. 30 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 30 depicts an illustrative computer architecture for a computer 1500 for practicing the various embodiments of the invention. The computer architecture shown in FIG. 30 illustrates a conventional personal computer, including a central processing unit 1550 (“CPU”), a system memory 1505, including a random-access memory 1510 (“RAM”) and a read-only memory (“ROM”) 1515, and a system bus 1535 that couples the system memory 1505 to the CPU 1550. A basic input/output system containing the basic routines that help to transfer information between elements within the computer, such as during startup, is stored in the ROM 1515. The computer 1500 further includes a storage device 1520 for storing an operating system 1525, application/program 1530, and data.

The storage device 1520 is connected to the CPU 1550 through a storage controller (not shown) connected to the bus 1535. The storage device 1520 and its associated computer-readable media, provide non-volatile storage for the computer 1500. Although the description of computer-readable media contained herein refers to a storage device, such as a hard disk or CD-ROM drive, it should be appreciated by those skilled in the art that computer-readable media can be any available media that can be accessed by the computer 1500.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 1500 may operate in a networked environment using logical connections to remote computers through a network 1540, such as TCP/IP network such as the Internet or an intranet. The computer 1500 may connect to the network 1540 through a network interface unit 1545 connected to the bus 1535. It should be appreciated that the network interface unit 1545 may also be utilized to connect to other types of networks and remote computer systems.

The computer 1500 may also include an input/output controller 1555 for receiving and processing input from a number of input/output devices 1560, including a keyboard, a mouse, a touchscreen, a camera, a microphone, a controller, a joystick, or other type of input device. Similarly, the input/output controller 1555 may provide output to a display screen, a printer, a speaker, or other type of output device. The computer 1500 can connect to the input/output device 1560 via a wired connection including, but not limited to, fiber optic, ethernet, or copper wire or wireless means including, but not limited to, Bluetooth, Near-Field Communication (NFC), infrared, or other suitable wired or wireless connections.

As mentioned briefly above, a number of program modules and data files may be stored in the storage device 1520 and RAM 1510 of the computer 1500, including an operating system 1525 suitable for controlling the operation of a networked computer. The storage device 1520 and RAM 1510 may also store one or more applications/programs 1530. In particular, the storage device 1520 and RAM 1510 may store an application/program 1530 for providing a variety of functionalities to a user. For instance, the application/program 1530 may comprise many types of programs such as a word processing application, a spreadsheet application, a desktop publishing application, a database application, a gaming application, internet browsing application, electronic mail application, messaging application, and the like. According to an embodiment of the present invention, the application/program 1530 comprises a multiple functionality software application for providing word processing functionality, slide presentation functionality, spreadsheet functionality, database functionality and the like. The computer 1500 in some embodiments can include a variety of sensors 1565 for monitoring the environment surrounding and the environment internal to the computer 1500. These sensors 1565 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

### NUMERATED EMBODIMENTS

Embodiment 1. A microbubble array comprising:
a microbubble membrane comprising a plurality of microbubbles, each microbubble of the plurality of microbubbles having a first opening and a second opening diametrically opposing the first opening, and
a nanoporous membrane positioned below the microbubble membrane, wherein a first cell type is positioned within at least a portion of the microbubbles of the plurality of microbubbles, and a second cell type is positioned on a bottom surface of the nanoporous membrane.

Embodiment 2. The microbubble array of embodiment 1, wherein the first cell type comprises at least one of salivary gland tissue cells, salivary gland cell clusters, or salivary gland cells.

Embodiment 3. The microbubble array of any previous embodiment, wherein the second cell type comprises endothelial cells.

Embodiment 4. The microbubble array of any previous embodiment, further comprising at least a third cell type on the bottom surface of the nanoporous membrane, wherein the third cell type comprises at least one of nerve cells or immune cells.

Embodiment 5. The microbubble array of any previous embodiment, wherein each microbubble of the plurality of microbubbles is at least partially filled with at least one of a polymer, a hydrogel, a polymer crosslinking agent, a hydrogel crosslinking agent.

Embodiment 6. The microbubble array of any previous embodiment, wherein the first opening of each microbubble of the plurality of microbubbles is larger than the second opening.

Embodiment 7. The microbubble array of any previous embodiment, wherein the plurality of microbubbles has a total number of microbubbles ranging between 6 and 5000 microbubbles.

Embodiment 8. The microbubble array of any previous embodiment, wherein the microbubble membrane is formed from polydimethylsiloxane (PDMS).

Embodiment 9. The microbubble array of any previous embodiment, wherein the nanoporous membrane is formed from silicon or silicon nitride.

Embodiment 10. The microbubble array of any previous embodiment, wherein each microbubble of the plurality of microbubbles has a diameter ranging between 20 microns and 2000 microns.

Embodiment 11. The microbubble array of any previous embodiment, wherein the first opening and the second opening of each microbubble of the plurality of microbubbles has a width ranging between 5 microns and 1000 microns.

Embodiment 12. The microbubble array of any previous embodiment, wherein the pores of the nanoporous membrane have width ranging between 1 nm and 10,000 nm.

Embodiment 13. The microbubble array of any previous embodiment, wherein each microbubble of the plurality of microbubbles has an inner volume ranging between 1 nL and 200 nL.

Embodiment 14. The microbubble array of any previous embodiment, wherein the thickness of the microbubble membrane ranges between 0.03 mm and 3 mm, and the thickness of the nanoporous membrane ranges between 50 nm and 500 nm.

Embodiment 15. The microbubble array of any previous embodiment, further comprising a spacing portion with a central opening positioned between the microbubble membrane and nanoporous membrane, wherein a gap with a volume is formed by the opening and the gap is at least partially filled with a polymer or hydrogel Embodiment 16. A microfluidic device, comprising:
a housing at least partially enclosing at least one microbubble array of any previous embodiment;
a top channel in the housing fluidly connected to the first opening of each microbubble of the plurality of microbubbles; and
a bottom channel in the housing fluidly connected through the pores of the nanoporous membrane to the second opening of each microbubble in the plurality of microbubbles, wherein one or more fluids may be flowed into the top channel and collected or analyzed from the bottom channel.

Embodiment 17. The microfluidic device of embodiment 16, further comprising at least one sensor positioned below the nanoporous membrane in the bottom channel, configured to assess cell secreted factors.

Embodiment 18. The microfluidic device of any one of embodiments 16-17, further comprising at least one of a sensor, optical sensor, and photonic array configured to optically interrogate each microbubble of the plurality of microbubbles.

Embodiment 19. The microfluidic device of any one of embodiments 16-18, further comprising at least one sensor coupled to the microbubble membrane, configured to measure the electrical resistance of the microbubble membrane.

Embodiment 20. The microfluidic device of any one of embodiments 16-19, wherein the one or more fluids comprise at least one of water, blood, solutions, solutions of drugs, solutions of therapeutics, buffers, stimulants, or combinations thereof.

Embodiment 21. The microfluidic device of any one of embodiments 16-20, wherein the device is configured to recapitulate one or more disease states, wherein the disease states are selected from tumors, salivary gland, eye diseases, lung diseases, kidney diseases, GI tract diseases, eye diseases, stomach diseases, or any other organ disease.

Embodiment 22. A microphysiological system, comprising:
a top portion having a body with at least one cavity, an inlet, and an outlet, each passing through the body;
at least one microbubble array of any one of embodiments 1-15 positioned within the at least one cavity;
a bottom portion comprising a plate having an indentation forming a channel, wherein the top portion is fixedly and removably attached to the bottom portion and the cavity, inlet and outlet are fluidly connected by the channel, and wherein one or more fluids may be flowed into the inlet, analyzed within the channel, and collected from the outlet.

Embodiment 23. The microphysiological system of embodiment 22, wherein the microbubble membrane is attached to the nanoporous membrane with a spacing portion forming a gap between the microbubble membrane and the nanoporous membrane.

Embodiment 24. The microphysiological system of any one of embodiments 22-23, wherein the spacing portion comprises a layer of pressure sensitive adhesive with a central opening passing through the layer.

Embodiment 25. The microphysiological system of any one of embodiments 22-24, wherein the gap is at least partially fluidly filled with a hydrogel.

Embodiment 26. The microphysiological system of any one of embodiments 22-25, wherein the hydrogel comprises at least one PEG, Matrigel, collagen, fibrinogen, alginate, polyacrylamide, and hyaluronic acid.

Embodiment 27. The microphysiological system of any one of embodiments 22-26, further comprising one or more sensors positioned within the top or bottom portion of the system.

Embodiment 28. The microphysiological system of any one of embodiments 22-27, wherein the one or more sensors are selected from the group consisting of: optical sensors, photonic arrays, transelectrical resistance (TEER) sensors, biosensors, flow rate sensors, fluid pressure sensors, temperature sensors, pH sensors, oxygen sensors, carbon dioxide sensors, and electrochemical sensors.

Embodiment 29. The microphysiological system of any one of embodiments 22-28, wherein the one or more fluids comprise at least one of cell culture media, water, blood, solutions, solutions of drugs, solutions of therapeutics, buffers, solutions of bioactive substances, stimulants, or combinations thereof.

Embodiment 30. The microphysiological system of any one of embodiments 22-29, wherein the device is configured to recapitulate one or more normal tissue or tissue disease states, wherein the disease states are selected from tumors, eye diseases, lung diseases, kidney diseases, stomach diseases, or diseases of any organ system.

Embodiment 31. A method of fabricating a microbubble membrane, comprising the steps of:
providing a template comprising an array of cavities;
placing a cover over the cavities of the template, the cover spaced away from the openings of the cavities with one or more spacers forming a gap between the template and cover;
pouring a precursor into the gap;
curing the precursor, thereby forming a plurality of microbubbles in the precursor;
removing the cover from the template;
removing the formed microbubble membrane from the template, the plurality of microbubbles each having a first opening; and
forming a second opening in each microbubble of the plurality of microbubbles.

Embodiment 32. A method of fabricating a microbubble membrane, comprising the steps of:
providing a surface;
pouring a precursor onto the surface;
providing a template comprising an array of cavities;
placing the template onto the precursor cavity side down;
curing the precursor, thereby forming a plurality of microbubbles in the precursor;
removing the template from the surface;
removing the formed microbubble membrane off the template, the plurality of microbubbles each having a first opening; and
forming a second opening in each microbubble of the plurality of microbubbles.

Embodiment 33. The method of embodiments 31 or 32, wherein the step of forming the second opening comprises at least one of using an acid solution to etch a portion of the membrane or remove an excess precursor layer, using a reactive ion gas phase etching process to etch a portion of the membrane or remove an excess precursor layer, using a laser ablation process to etch cylindrical openings in the bottom surface of the membrane, or using a vibratome to remove an excess precursor layer.

Embodiment 34. The method of claim any one of embodiment 31, wherein the spacer has a height ranging between 0.1 mm and 10 mm.

Embodiment 35. The method of any one of embodiments 31-34, wherein the precursor comprises PDMS, and the curing step comprises heating the precursor for a period of time.

Embodiment 36. A method of seeding a microbubble array, comprising the steps of:
providing the microbubble array of any one of embodiments 1-15;
providing a solution comprising the first cell type mixed with a hydrogel and crosslinking agent; and
flowing the solution into the top openings of at least a portion of the microbubbles of the microbubble membrane.

Embodiment 37: The method of embodiment 36, further comprising a step of polymerizing the solution such that the first cell type is embedded within a cross-linked hydrogel in at least a portion of the microbubbles.

Embodiment 37. The method of embodiment 36, wherein the crosslinking comprises photo crosslinking, thermal crosslinking, or chemical crosslinking.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Multi-Organ on a Chip in a Double-Sided Open Microbubble Array Coupled with Silicon Nanomembrane in a Microfluidic Device—Fabrication Method and Device

The GEM or iGEM molding process used to fabricate MB arrays is illustrated in FIGS. 7A and 7B and FIGS. 11A and 11B. The schematic shows the use of a silicon template for molding but it is obvious to those skilled in the art that other templates fabricated out of polymers (e.g. PDMS, photoresists) or other inorganic materials (glass, nitride, metals) would suffice for GEM molding. It is also obvious that 3D printing technologies (ink jet, laser or UVR forming etc.) could be used to directly fabricate curvilinear cavities but these methods are inefficient, costly, lack fidelity on the micron-scale and are limited by the use of certain materials and solvents that may be toxic for biological applications. Microbubble formation is a delicate balance of buoyancy force and surface tension. Air dissolved in the PDMS premix is essential for bubble growth and size of the MBs formed depends on the diameter of the pit and the thickness of the PDMS film poured onto the template. If the film is too thick, the MBs will grow too big, merge or boil off. Size is constrained by inter-pit (or well) spacing on the template.

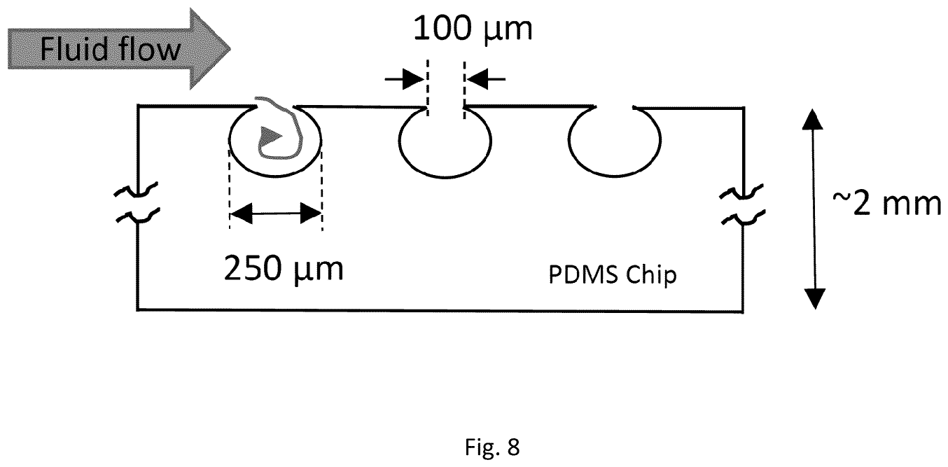

The GEM molding process (FIG. 7B) produces an array of MBs formed in a PDMS chip (Giang, U. B. et al. Biomed Microdevices 16, 55-67 (2014)). A 2D schematic (not to scale) of a MB array is shown in FIG. 8. The schematic shows a chip comprised of MBs with 100 μm diameter opening and a 250 μm MB diameter width (˜8 nL volume). The MB cavities typically project into the PDMS chip less than ˜ 1/10^{th} of the typical chip thickness (˜2 mm). The circular MB architecture is advantageous for cell and tissue culture. Structures designed with an aspect ratio (MB diameter/MB opening) of 2.5 allow nutrient and waste exchange, preventing lactate build up, while allowing for the concentration of cell secreted factors that can positively affect the biology. Microbubble arrays are comprised of a customizable high density of individual MBs, ranging from a few <50 MBs/cm² to many >4500 MBs/cm².

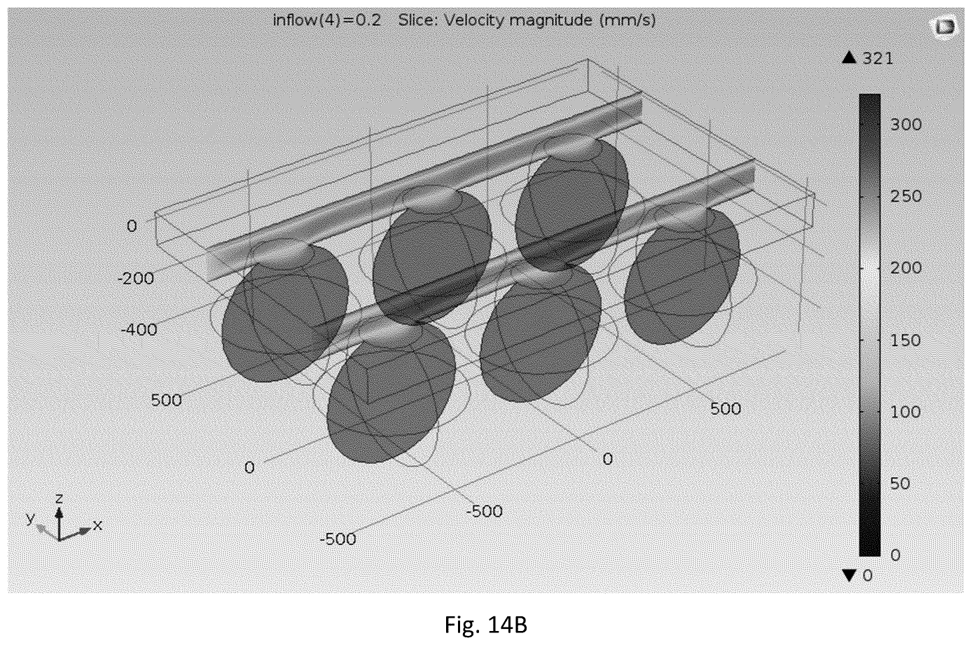

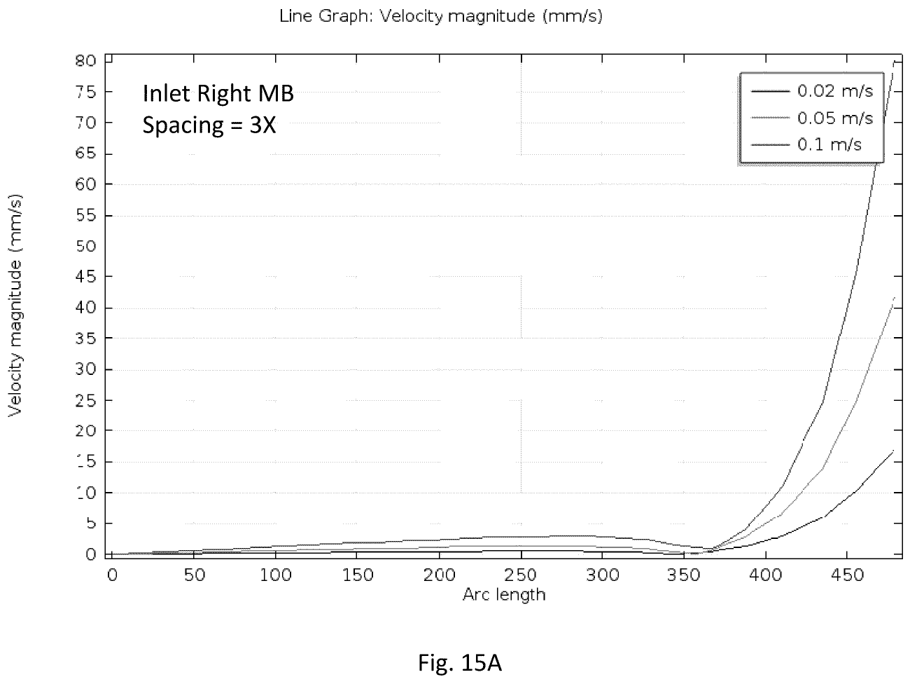

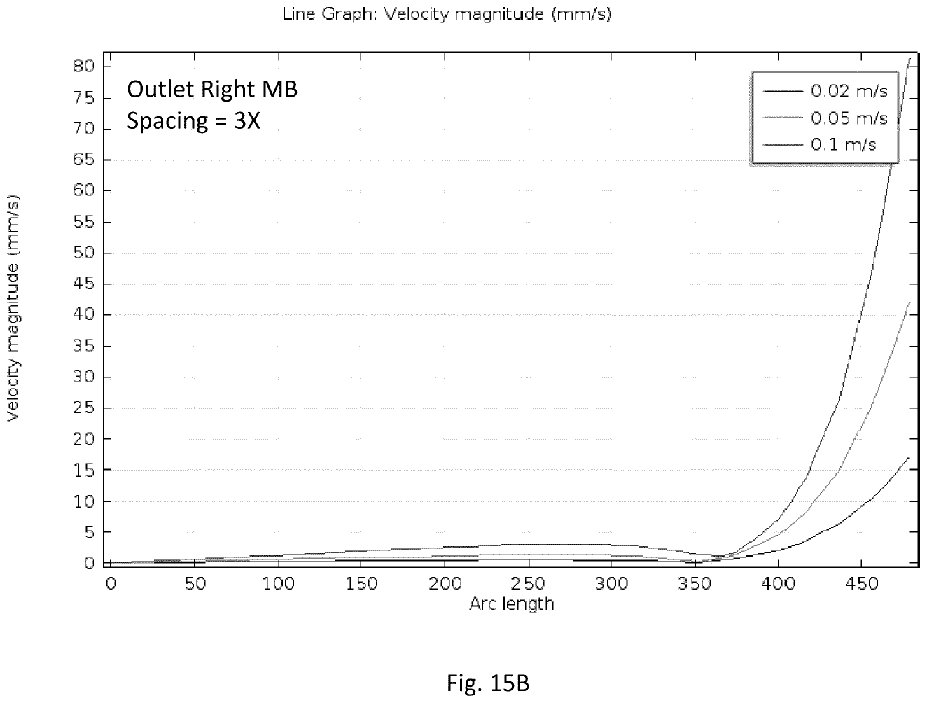

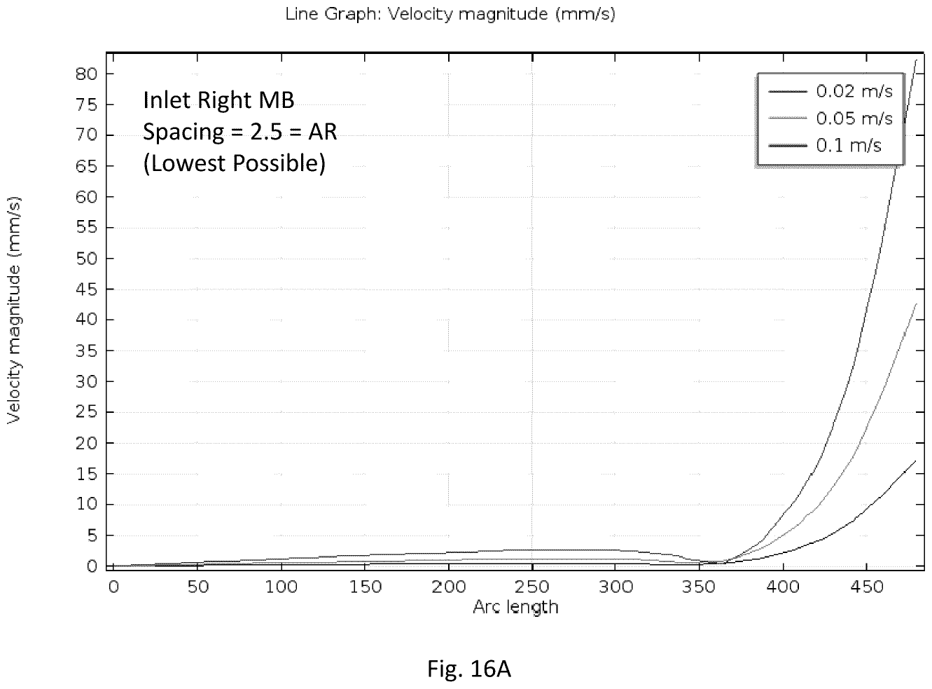

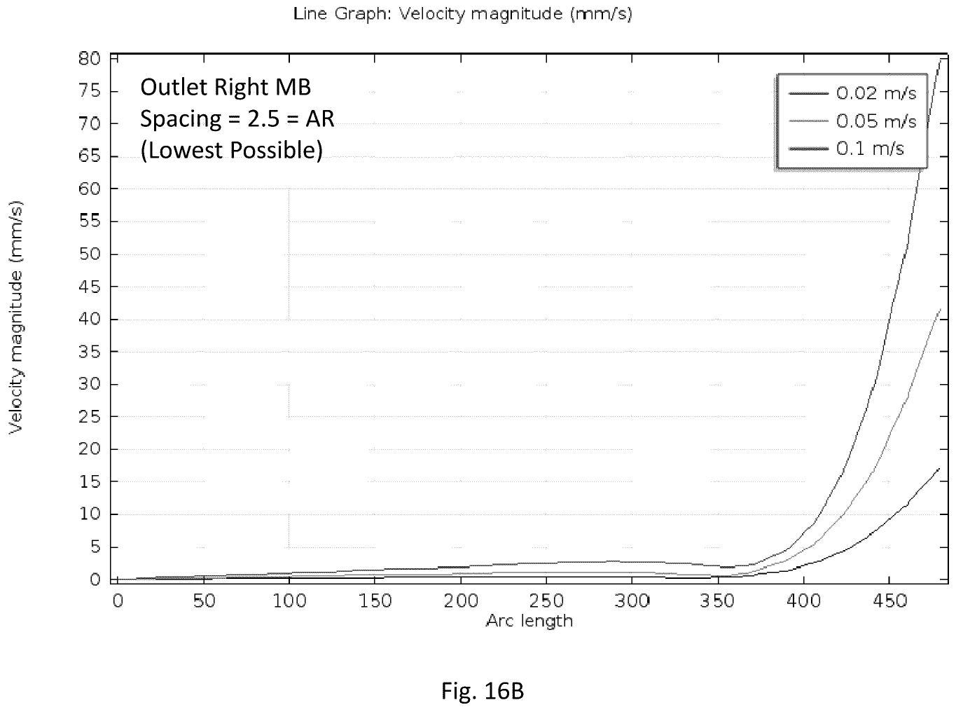

MB arrays are used in static cell culture or they can be mounted into a microfluidic systems or devices where fluid flows in a channel constructed above the MB cavities (Agastin, S. et al. Biomicrofluidics 5, 24110 (2011).; Bobo, B. et al. Lab Chip 14, 3640-3650 (2014).; Chandrasekaran, S. & DeLouise, L. Biomaterials 32, 9316-9327 (2011).; Chandrasekaran, S. et al. Biomaterials 32, 7159-7168 (2011).; Jones, M. C. et al. Biomed Microdevices 15, 453-463 (2013).; Pu, Q. et al. Biomed Microdevices 19, 17 (2017)). Microfluidic devices can be designed to allow and control fluid flow fields that enter into the MB cavities to introduce mixing and shear stress that may be studied on cells growing in each MB in the array. COMSOL simulation studies find that MBs spaced apart by >2.5× the diameter of the MB opening, experience independent fluid flow, the flow fields are not coupled (see FIGS. 14A-16B).

Biomedical applications and initial commercial sales of these arrays target applications in high throughput screening (HTS) of single cells and tissues for therapeutics (drugs and biologics) and cancer stem cell discovery. Salivary gland tissue chips were developed and used in static culture to discover radioprotective drugs (Piraino, Lindsay R et al. “Salivary Gland Tissue Engineering Approaches: State of the Art and Future Directions.” Cells vol. 10,7 1723. 8 Jul. 2021, doi:10.3390/cells10071723). Microbubble arrays (Nidus MB Technologies) are packaged into many common formats (multiwall plates and chamber slides).

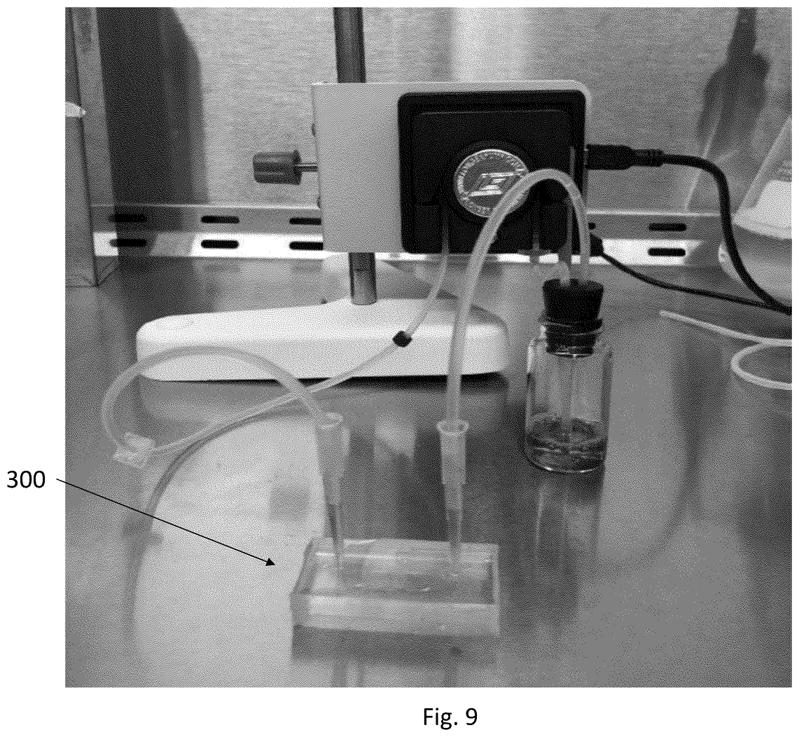

On-going research has successfully integrated single-side open MB arrays into a microfluidic device for salivary gland organoid culture (FIG. 9). Lamellar streams of fluid flow can be defined for high-throughput screening (HTS) studies of a drug concentration gradient across the chip or multiple drugs in single chip with each lamellar stream allowing analysis of many MB wells. PDMS is optically clear which allows in situ bright field and fluorescence imaging of cells growing in each MB. Addressable optical sensors can be integrated in this chip format to probe individual MB based on optical signals that transmit through the PDMS chip. This architecture, while quite versatile—enables numerous types of HTS and cell culture studies, however, it is limited in its capacity to couple to a second tissue compartment to create a microphysiological system (MPS). MPS or so-called organs-on-a-chip, couple many cell types in a device to more appropriately recapitulate a biological system. To overcome this limitation, disclosed herein is a method to fabricate a MB membrane and various embodiments comprising the membrane for tissue chip and MPS applications.

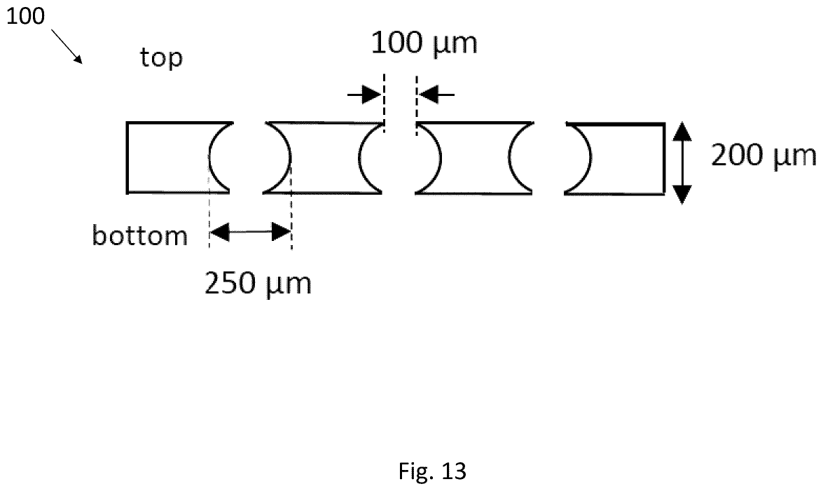

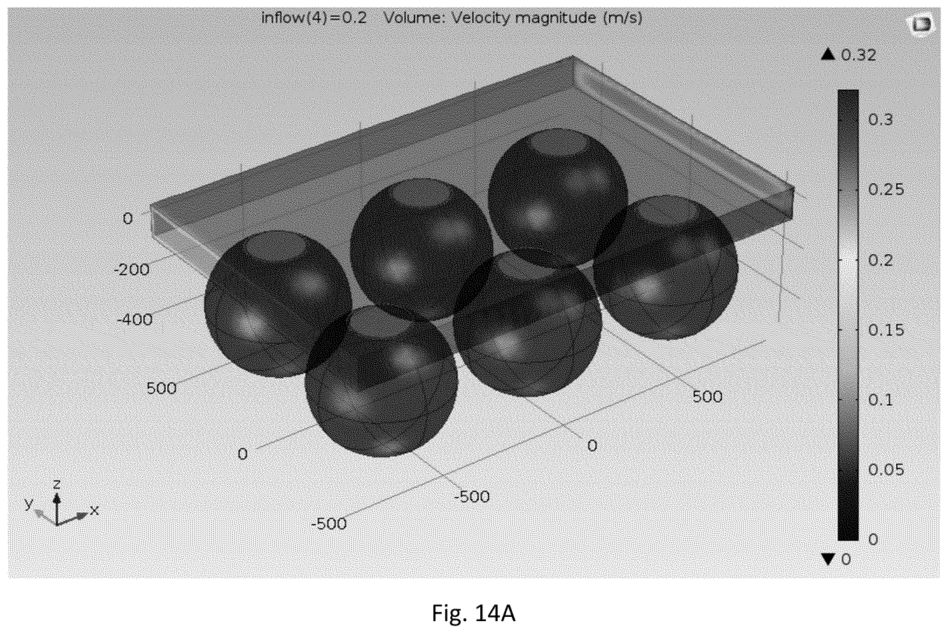

FIG. 13 depicts a schematic (not to scale) of an exemplary MB array membrane. Actual sizes in MB openings vary (20-500 micron diameter) and membrane thickness will vary accordingly. The key feature is the dual openings into the MB; one on the top and the other on the bottom of the MB membrane array. This dual opening architecture will enable coupling of the MB array membrane to other components to build microfluidic devices and MPS.

There are many methods to create the MB membrane:

Laser Ablation: It may be obvious to those skilled in the art that many methods could be used to open holes on the bottom side of a standard MB chip such laser ablation (FIG. 12). Such a process however, would be time consuming and expensive. Moreover, this method would create deep cylindrical openings through the thick PDMS chip. These would create diffusion gradients that would hinder exchange of secreted products from cells cultured on opposite sides of the membrane and it would restrict cell-cell contacts from forming. FIG. 12 depicts a schematic of laser ablation of deep cylindrical opening from the underside of a standard MB chip. Not to scale.

Restricted Gas Expansion Molding: A variation of the GEM process called restricted gas expansion molding (rGEM) has been developed to fabricate thin microbubble membrane arrays. This process is illustrated in FIGS. 10A and 10B.

FIG. 10B depicts a schematic (not to scale) of the restricted rGEM molding process developed to mold microbubble membranes. Spacers and a cover are placed on the template. The spacers are used to restrict the growth of the microbubble that form over the cavities or pits. PDMS is poured in the gap space. Upon curing, the MBs form but their growth/expansion is restricted by the cover. After curing, when the cover is removed, it is evident that an opening is formed when the expanding bubble presses against the cover. The process of removing the covers remove any PDMS skin that forms. An oxygen plasma can also be used to further clean a residual polymer.

Spacers and a cover are placed on top of the template. The spacer height is critical to restrict the microbubble growth to be less than the extent that would normally form. A PDMS film is poured into the gap space over the template as in the normal GEM process. The cover restricts bubble expansion. When cured, a hole is formed where the bubble contacted the cover. The cover can be comprised of many materials including glass, silicon, Teflon. It is possible that a thin PDMS skin may form during expansion but this will be ripped off when the cover is removed. The cover can be treated to ensure an opening forms when the cover is removed.

Upside-Down Casting: Upside-down casting is a method to fabricate thin MB arrays with a particular thickness (FIGS. 11A and 11B). The concept is that uncured PDMS is poured into a plate and the template is placed upside down on the PDMS. Knowing the surface area of a plate, the exact volume of PDMS premix is added to achieve a thin microbubble array of a particular thickness. The target thickness depends on the size of the intended MBs plus ˜10% more. For example, if the MBs were to be 300 μm diameter the total thickness target would be 330 m. Molding upside down has the advantage of the premix not flowing into templates that are designed to make large diameter (>400 μm) MB openings. Moreover, it overcomes the effects of buoyance, in making larger MBs, where surface tension might not hold the MB to the template prior to curing. Although this method allows for the manufacture of thin membranes, the 10% extra thickness requires a process to open the hole on the backside. Laser ablation can be used as described above with the already mentioned negative attributes. There are two preferred methods of creating the second opening in the MB.

The excess layer of PDMS can be chemically etched by immersing the membrane in a sulfuric acid 98% solution until the bottom openings are created (double-sided open). This causes pitting of the PDMS that effects optical clarity. Alternatively, a reactive ion gas phase etching process (RIE) can be used by optimizing the combination of O₂, SF₆, and CHF₃, to remove the excess PDMS layer. The RIE process is however slow and costly. The vibratome is the preferred method of making the second opening as it is a rapid process that can be controlled to produce the desired opening size.

The two-side open MB membrane architecture overcomes limitations associated with integrating a standard MB array chip into microfluidic devices, MPS and other tissue chip systems or devices. Such systems are generally composed of fluid channels, pumps, sensors, and supports. One embodiment comprises the integration of a nanosilicon membrane support. Nanosilicon membranes are porous materials on nanometer thickness and comprise a technology platform produced and sold by SiMPore, Inc. An example of a composite device is illustrated in FIG. 1A. This composite architecture will allow implementation of an MPS that integrates two organ components; one grown in the MB wells (e.g salivary gland tissue) and the other grown on the underside of the nanoporous silicon membrane (endothelial, nerves or immune cells) (FIG. 1B). The benefits of using the nanoporous membrane as a cell support are well described in literature published by the McGrath Lab (Burgin, T. et al. Membranes (Basel) 6 (2015).; Burgin, T. et al. Proc Int Conf Nanochannels Microchannels Minichannels 2016 (2016).; Dehghani, M. et al. Adv Mater Technol 4 (2019).; Khire, T. S. et al. Biomed Microdevices 20, 11 (2018).; Khire, T. S. et al. Cell Mol Bioeng 13, 125-139 (2020).; Kim, E. et al. J Am Chem Soc 130, 4230-4231 (2008).; Masters, E. A. et al. Nanomedicine 21, 102039 (2019).; Mossu, A. et al. J Cereb Blood Flow Metab 39, 395-410 (2019)). The foremost advantages are 1. optical transparency enabling imaging on both sides of the nanomembrane, 2. resistance free transport of soluble bioactive factors between the cell compartments and 3. the direct cell-to-cell contact that can occur via cell transmigration or for example, extension of neuron dendrites or microvessels through the pores specifically engineered in the nanomembrane. A double-sided open MB array is coupled to the nanosilicon membrane (hybrid platform) using oxygen plasma treatment or a pressure sensitive adhesive (PSA) under optimal conditions.

FIG. 1A depicts a schematic (not to scale) of a MB array membrane array coupled to a nanoporous silicon membrane. FIG. 1B depicts a schematic (not to scale) of the combined membrane MPS with co-cultured brown cells in the MB and green cells culture on the nanoporous silicon membrane. A second embodiment is integration of the membrane into a microfluidic device. In the next step, the hybrid platform is integrated into a microfluidic device called μSiM (microphysiological system or microfluidic device enabled by a silicon membrane). A prototype of the disclosed device is shown in FIGS. 5A-6. The nanometer thick (100-400 nm) silicone membrane in the μSiM device plays a critical role in multi-organ development as it provides reasonably resistance-free paracrine signaling between the cells/tissues culture in the MB on the top side and the other cells/tissues cultured on the underside. The model with enhanced complexity can bridge the gap between in vitro cell culture models and in vivo responses by more precisely recapitulating the native tissue and cellular responses while reducing the costs and ethical concerns regarding animal use (Wanigasekara, J. et al. Plos one 18, e0276248 (2023).; Cacciamali, A. et al. Frontiers in Physiology 13, 836480 (2022)).

FIG. 2 depicts an exemplary microfluidic device where fluid containing drug or stimulating factors is flowed over the cells and cell secreted factors that are collected in the basal compartment.

The example shown in FIG. 2 illustrates mounting the MB membrane in a microfluidic device. The fluid may contain media or media plus factors (drugs, stimulants etc.) and the effect on cell secreted factors can be measure on the back side. FIG. 3 depicts the microbubble membrane coupled to a nanoporous membrane and fluid flow established on a top side of the microbubble membrane and on a bottom side of the nanoporous membrane.

A third embodiment would be to integrate an optical sensor or photonic array into the device for assessing cell secreted factors or to optically interrogate each well independently. FIG. 4 depicts an exemplary integrated optical sensor into the microfluidic device for in situ biosensing.

A fourth embodiment would be to couple the MB membrane to an electrical device to measure resistance or barrier function of the cells, or to stimulate cells (e.g. neurons) or tissue growing under fluid flow.

According to the FDA Modernization Act 2.0, drug testing in animals is no longer mandatory; that is, preclinical tests (including tests on animals) can be replaced with alternatives like in vitro and in silico experiments in the forms of MPS and computer modeling that are more scientifically relevant (Adashi, E. Y. et al. The American Journal of Medicine (2023)). The platform described has the promising potential for progression to qualification through the FDA as a drug development tool to reduce animal use for drug development and to be employed for different tissue cultures like primary tumors for precision medicine. While replicating native tissue, the platform will enable in situ biosensing of the tissue function and high throughput drug screening in a single microfluidic device.

### Example 2: Development of Functional Multi-Organ-on-a-Chip

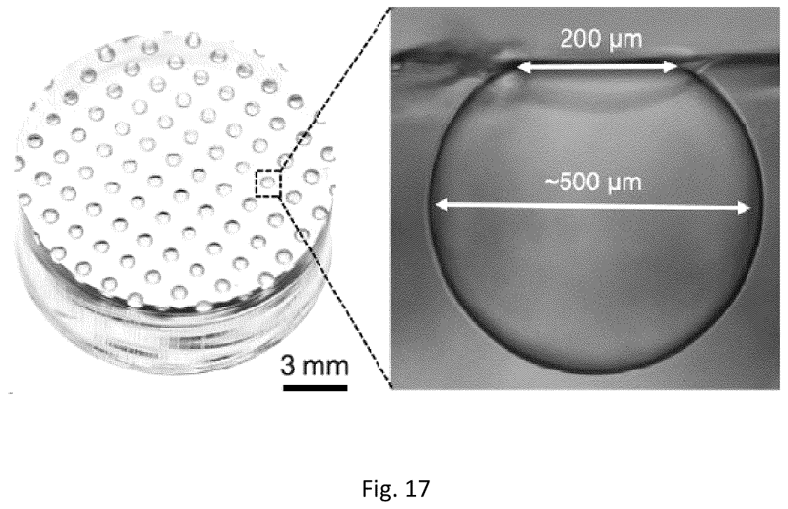

Salivary glands (SGs) are essential organs that produce and secrete saliva in the oral cavity. Radiotherapy for head and neck cancer treatment damages the SGs resulting in adverse side effects including oral infections, difficulty in speaking and eating due to lack of saliva. Limited progress in disease modeling and drug development for SG dysfunction is associated with the lack of functional in vitro SG tissue mimetics (SGm). To address this, the polydimethylsiloxane (PDMS) microbubble (MB) array platform with multiple microwells (˜250 MB/cm²) can be utilized for SGm formation (FIGS. 17-20) in a high throughput manner with up to a 120-fold reduction in mouse usage in in vivo drug screening experiments. SG cells were seeded in a tunable matrix metalloproteinase (MMP)-degradable poly(ethylene glycol) (PEG) hydrogel and cultured under static conditions. The architecture of the MB enables autocrine/paracrine signaling, and long-term culture, and allows in situ imaging.

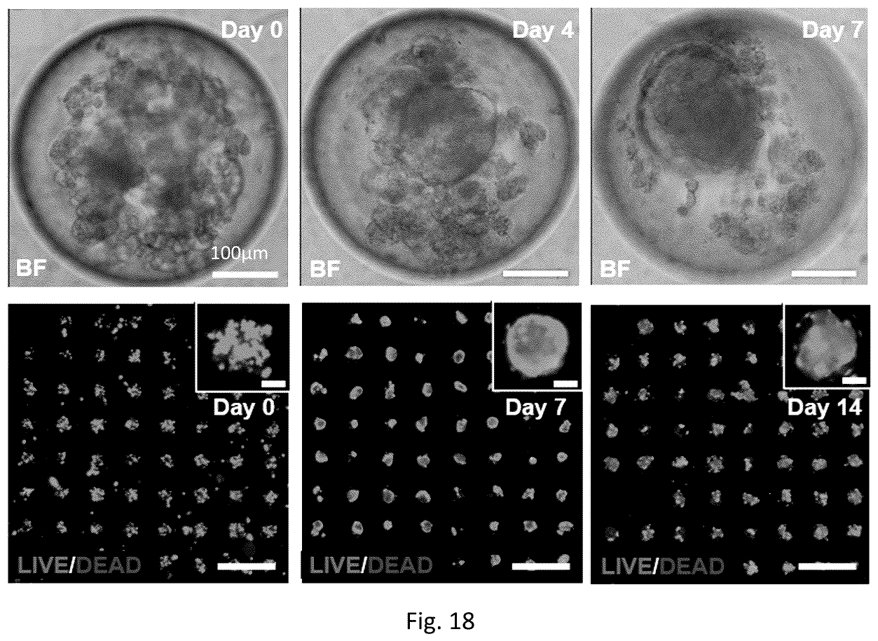

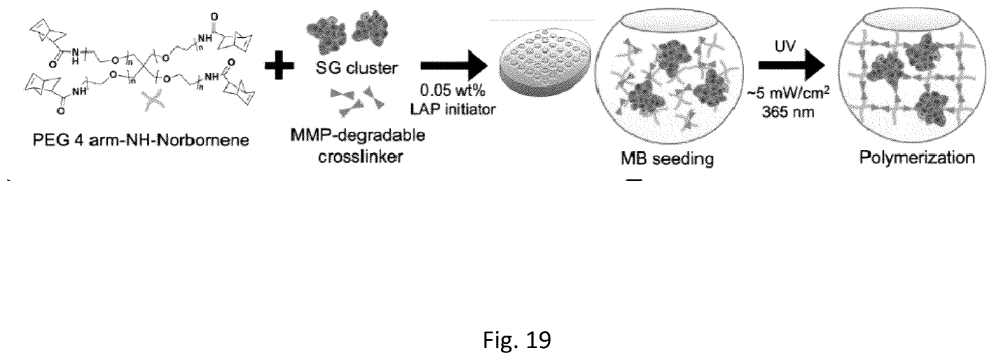

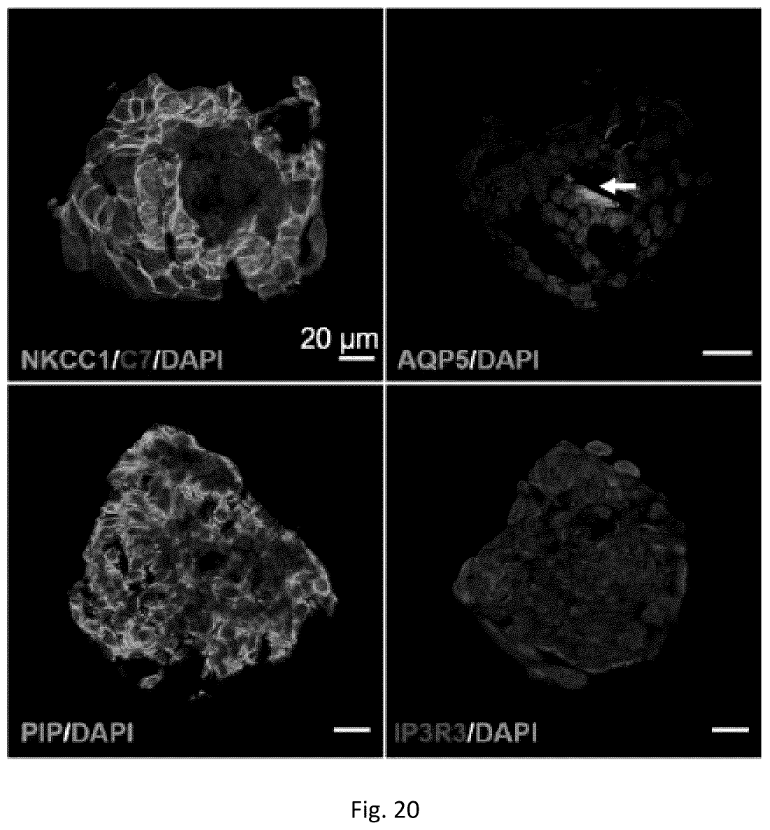

FIG. 17 depicts an exemplary MB-chip and a cross-sectional view of a single MB. FIG. 19 is a schematic showing an exemplary representation of hydrogel encapsulation of SG cluster cells within MB-chips. FIG. 18 depicts exemplary Brightfield images of SGm formation in MB-chips at days 0, 4, and 7 and exemplary images of fluorescent LIVE (green) and DEAD (red) staining of cells in MB chips at Day 0, Day 7, and Day 14. (scale bars=1 mm, inserts=100 μm). FIG. 20 depicts exemplary images of IHC staining for NKCC1, PIP, IP3R3, and DAPI within SGm at day 14. The chips depicted in FIG. 17 were successfully used to discover novel radioprotective drugs (Song, Yuanhui et al. “Development of a functional salivary gland tissue chip with potential for high-content drug screening.” Communications biology vol. 4,1 361. 19 Mar. 2021.)

Objectives: This disclosure aims to design a microphysiological system (MPS) for the integration of MB chips and employ that for dynamic SGm culture. This disclosure also aims to develop a state-of-the-art MPS capable of culturing multi-organ-on-a-chip. Specifically, for building the first ever MPS to culture innervated vascularized 3D SGm in an arrayed format for high-content drug delivery.

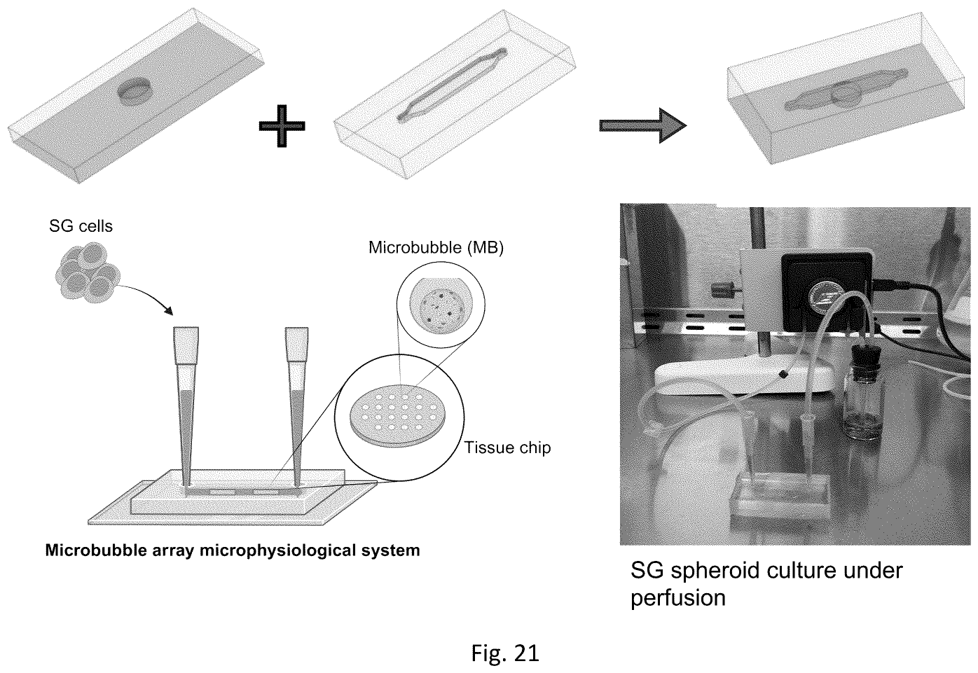

Methods: A two-layer microfluidic device was prepared for dynamic SGm culture (FIG. 21). The bottom layer accommodates the MB arrays and the top layer has a channel architecture for cell and fluid flow introduction. Introducing extracted SG cells from a mouse, SGm were cultured in the MB arrays under flow.

To advance the model and reliably recapitulate the SG tissue microenvironment complex, a microphysiological device for SGm culture capable of microfluidic perfusion and multi-organ integration was developed. A two-side opened MB array was bonded to an ultra-thin microporous silicon nitride (SiN) nanomembrane (400 nm thick) using a pressure sensitive adhesive (PSA) and rapidly assembled in a companion microfluidic silicon membrane cartridge called the micro-SiM (pSiM) (FIGS. 5A-5D). Using a PSA between the MB array and SiN membrane, a gap was created to be filled with hydrogel and exploited for the engineering of the tissue microenvironment. This disclosure combines expertise in MB array, hydrogel-based extracellular matrix, and SiN nanomembrane microfluidic technologies.

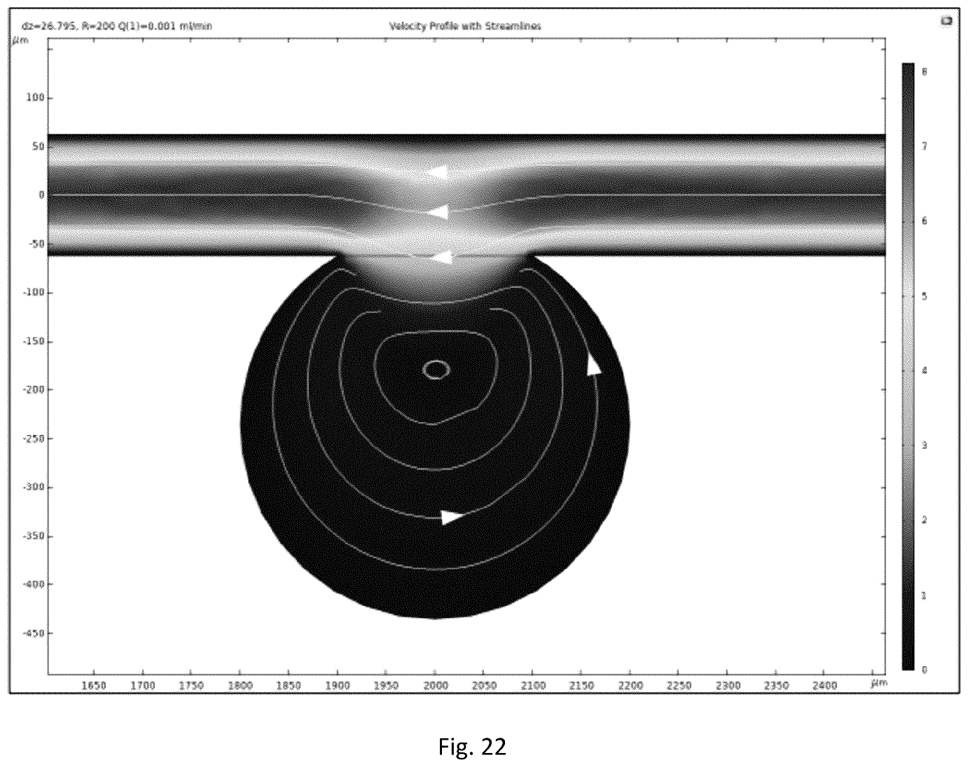

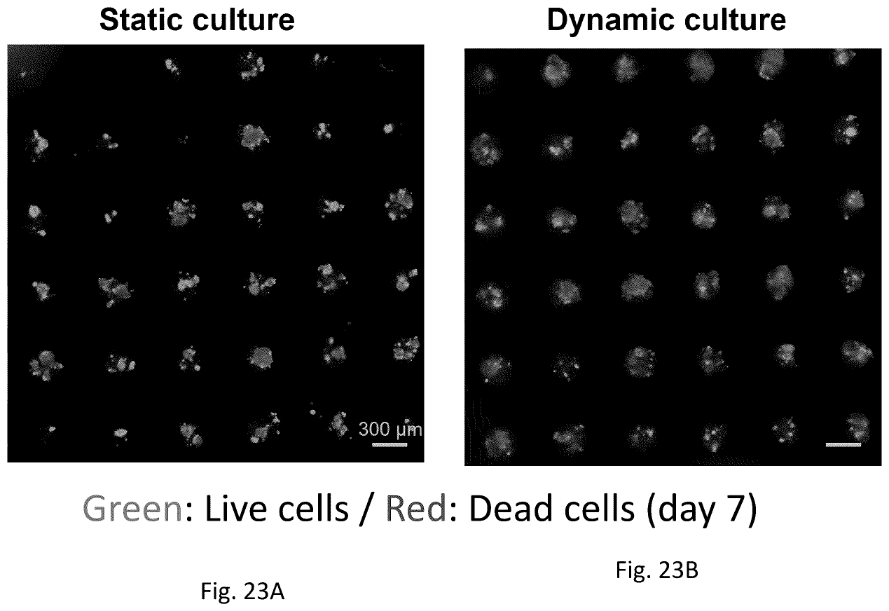

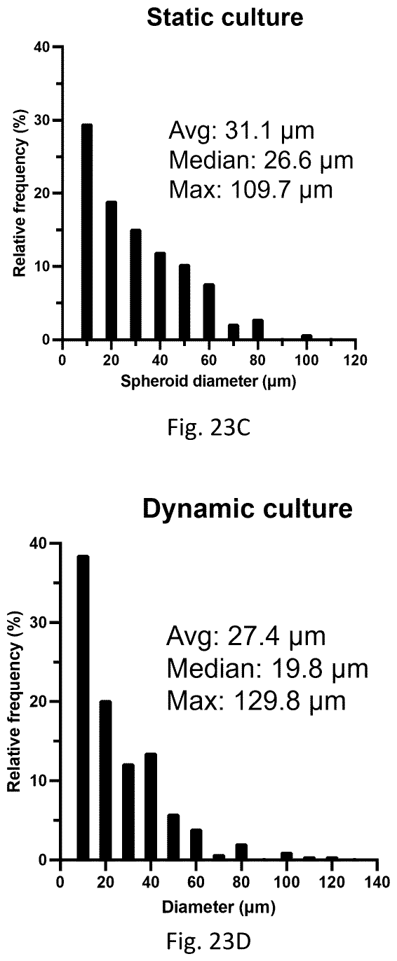

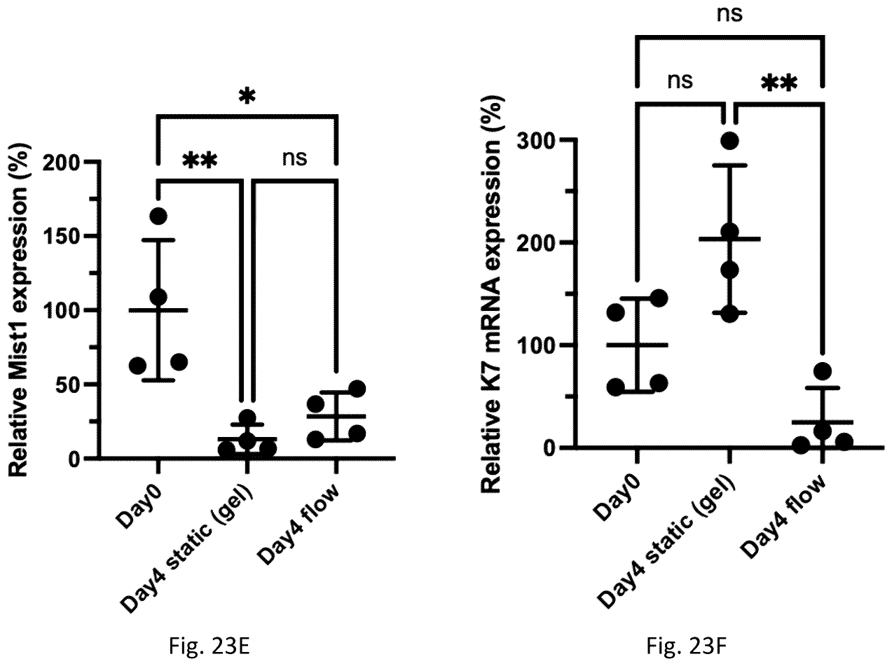

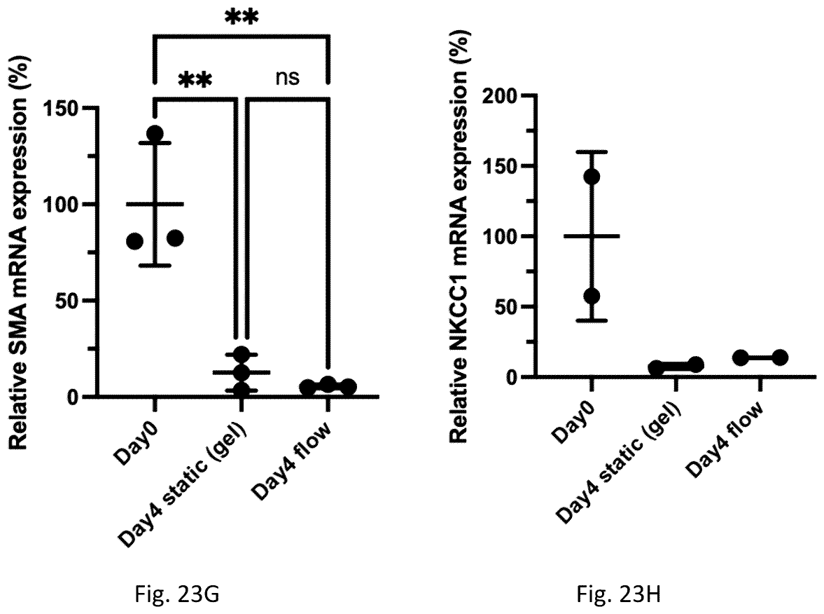

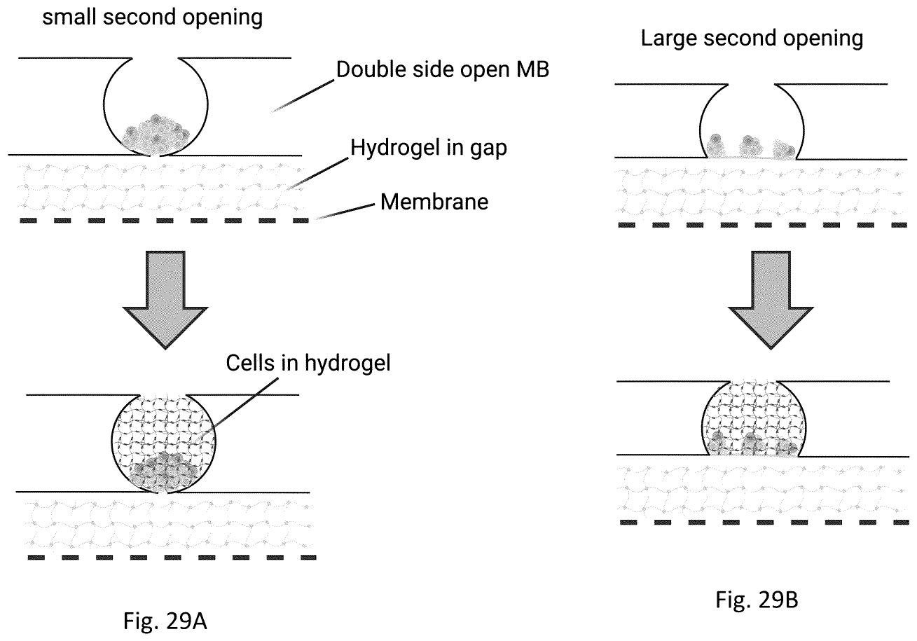

Results: COMSOL simulation in the developed MPS device demonstrated that cells in the MB arrays are not under direct shear stress from the fluid over it (FIG. 22). However, the fluid flow can accelerate media exchange in the MB, provide nutrients for the cells, and remove waste from their microenvironment. SGm were maintained alive under flow for over 7 days (FIGS. 23A and 23B). This was the first time that SG tissue has been cultured under flow in vitro. Similar size and morphology were observed for the SGm for both dynamic and static cultures (FIGS. 23C and 23D). FIG. 23E is a plot of exemplary qPCR results showing relative Mist1 expression against time for an SGm culture. FIG. 23F is a plot of exemplary qPCR results depicting relative K7 mRNA expression against time for an SGm culture. FIG. 23G is a plot of exemplary qPCR results depicting relative SMA mRNA expression against time for an SGm culture. FIG. 23H is a plot of exemplary qPCR results depicting relative NKCC1 mRNA expression against time for an SGm culture. FIG. 23I is a plot of exemplary qPCR results depicting relative Lyz2 mRNA expression against time for an SGm culture. FIGS. 29A and 29B depict schematics of exemplary MB arrays with small and large bottom openings.

Conclusions: 1) single-side open microbubble tissue chips support 3D spheroid/organoid long-term growth, phenotype retention, and high throughput drug screening with defined biomarkers. 2) SGm cultured for the first time under flow can better recapitulate in vivo conditions compared to static culture. 3) The modular pSiM utilizes mass-produced components to allow quick assembly of the multi-organ MPS. 4) The innovative hybrid μSiM platform can enable multi-organ-on-a-chip (SG with neuron/vasculature system) development and serve as a model for various diseases impacting SGs like Sjögren's syndrome, accelerating the development of new therapies.

### Example 3: Development of Functional Multi-Organ-on-a-Chip for Salivary Gland Disease Modeling and High-Throughput Drug Discovery

Salivary glands structure and function: There are three major SGs including parotid, submandibular, and sublingual glands. The general structure of SG consists of acinar cells, myoepithelial cells and ductal cells. The saliva produced by acinar cells is conducted by ductal structure to the mouth and has enzymes, electrolytes, mucins, and other components that contribute to different activities like digestion, antibacterial activity, and lubrication.

Radiotherapy: Radiotherapy, widely used for head and neck cancer treatment, leads to salivary gland (SG) dysfunction and xerostomia (i.e., dry mouth). Radiotherapy for head and neck cancer is given annually to around 1 million new patients worldwide. Radiotherapy has side effects, including salivary gland dysfunction. Some symptoms include oral infections, gum disease, tooth decay, difficulty in speaking, eating, and swallowing. Radiotherapy that is widely used for head and neck cancer treatment can severely damage salivary glands and lead to dysfunction with the symptoms of oral infections, gum disease, tooth decay, difficulty in speaking, eating, and swallowing.

Microbubble tissue chips: Microbubble tissue chip model supports cell viability in 3D tissue microenvironment and can reduce the use of animal models. Tissue chip with an array of microbubbles is made of PDMS, and is used for 3D cell culture. There are 40-50 MBs on one chip with MB density of 285 MB/cm² (FIG. 17). It's been demonstrated that tissue chips can support cell viability and SG tissue mimetic formation in a high-throughput manner with up to 120-fold reduction in mouse usage in in vivo drug screening experiments. In fact, by extracting salivary gland cells from one mouse, we can screen 40 different drugs, each with 50 replicates. Otherwise, 120 mice would be needed for screening of 40 drugs with the number of replication of 3. Development of this model was a significant improvement but still not perfect. FIG. 20 depicts fluorescence microscopy images of salivary gland tissue chips (Song, Y et al. Communications Biology, 2021). It has also been attempted on human SG however it is hard to obtain primary healthy human salivary gland tissue. In general, more than 80% of drugs fail in clinical trial stages and cannot get FDA approval due to the inadequacy of the models. So more physiologically relevant systems are needed for in vitro salivary gland replication. The key point in salivary gland tissue engineering is retaining the acinar cells phenotype and function after extraction from donors which are responsible for producing saliva. So salivary gland mimetics (SGm) were cultured in PEG hydrogel in microbubble arrays. Mereness et al. (Mereness, J A et al. Acta Biomaterialia, 2023) optimized media conditions and hydrogel degradation respectively and showed increased expression of acinar cell markers and better response to calcium stimulation, but there is still room for improvement.

The salivary gland microenvironment is complex and there is a lack of comprehensive in vitro models to study SG. There have been some improvements in tissue replication of the main structure and function, but there are a number of other components that have functions in supporting radio protection. The SG microenvironment is majorly comprised of acinar cells, ductal cells, myoepithelial cells, complex autonomic innervation, vasculature, immune cells, and ECM, where neurons stimulate myoepithelial and consequently acinar cells to produce saliva which goes through ductal structure toward the mouth cavity. The goal is to incorporate these major components into a single model for drug testing. For example, if a potential drug preserves nerve function it may thereby improve saliva production process. (Chibly, A M., et al. Physiological reviews, 2022).

Organ-on-a-chip models: Organ-on-a-Chip models can provide us with 3-dimensional tissue architecture under biorelevant conditions. They can enable high throughput drug screening and multi-tissue development with in-situ biosensing. They have a huge potential to reduce animal use in drug development and screening. They can recapitulate diseases, enable high-throughput drug screening, and reduce the need for animal use. Other benefits may include spatial-temporal control, ability of perfusion culture, mechanical cues modeling, high-throughput analysis with more reliable drug screening results, multi-tissues or organs interaction, integration of biosensing and bioimaging, reduction use of animal models, and closer features to in vivo natural systems. (Fang, G et al. Advanced Functional Materials, 2023)

SG culture: Under static conditions, successful SGm formation and tissue function was demonstrated. The goal was to enhance the system with microfluidics for better tissue microenvironment modeling. A microfluidic device was designed for the integration of single-side open MB array chips in a bottom layer with a top channel over it for the introduction of cells, media and fluid flow. The device was prepared and SGms were cultured under perfusion. FIG. 21 shows a schematic of the microfluidic device under flow, the integration of its component parts, and an image of SG spheroid culture under perfusion. It was found that SG dynamic culture may better preserve the native gland in terms of the acinar to duct cell ratio. COMSOL simulation of the microfluidic device demonstrated that cells in the MBs are not under direct shear stress from the fluid over the MB array acts like the vasculature for tissues and assist in exchanging the culture media to provide nutrients for the cells, and remove waste from their microenvironment (FIG. 22). The SGm was cultured under perfusion and maintained alive for over 7 days. Similar size and morphology were observed for the SGm for both dynamic and static cultures (FIGS. 23A and 23B). However, preliminary qPCR data showed a trend that suggests that promotion of acinar and decrease of K7 phenotype in dynamic culture (FIGS. 23C-23I). The goal was to have the gene expression as close as possible to the day 0 after extraction of cells from mice.

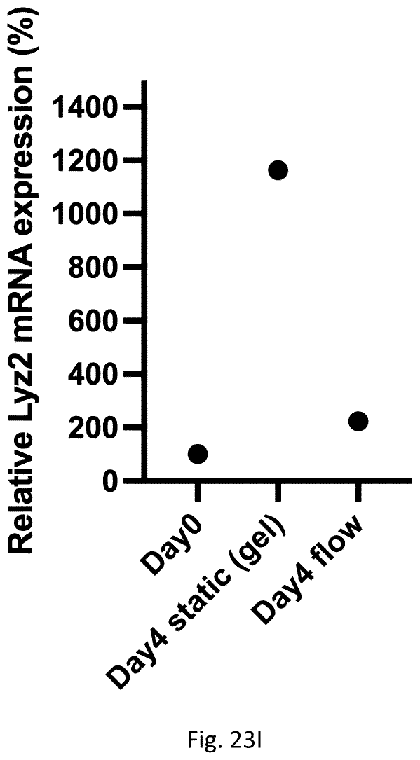

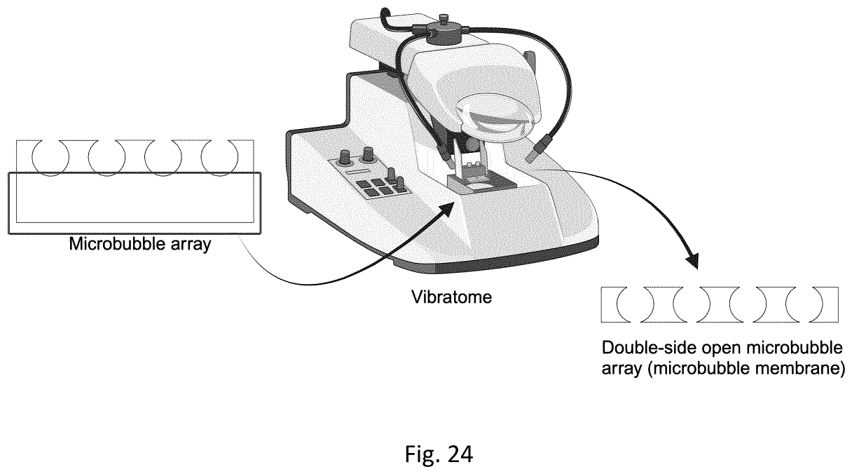

Hybrid Multi-Organ-on-a-Chip Model for Advanced Disease Modeling: In order to further advance the model and replicate in vivogland, a second and/or third tissue needed to be added to SGm including vasculature and nerve system. The MBs were opened on both sides, and a nanomembrane was bonded using a PSA to connect the SGm to other tissues and put under flow (FIG. 3). A vibratome was used to cut the bottom part of the array and create the second openings (FIG. 24).

Integration of components in the hybrid μSiM device for the development of multi-organ-on-a-chip: Then the produced double-side opened MB array bonded to a nanomembrane was integrated into a manufactured uSim device. (FIGS. 5A-5D). A pressure sensitive adhesive layer is sandwiched between the MB array and the nanomembrane to bind them together and make a gap equal to the thickness to the PSA that can be tailored to the specific application. Here a gap (˜125 μm thick) was created for the engineering of a specific tissue microenvironment. As it only covers the frame of the membrane, it creates a gap that can be exploited for better replication of in vivo conditions. FIG. 6 shows an image of an exemplary prototype of the device.

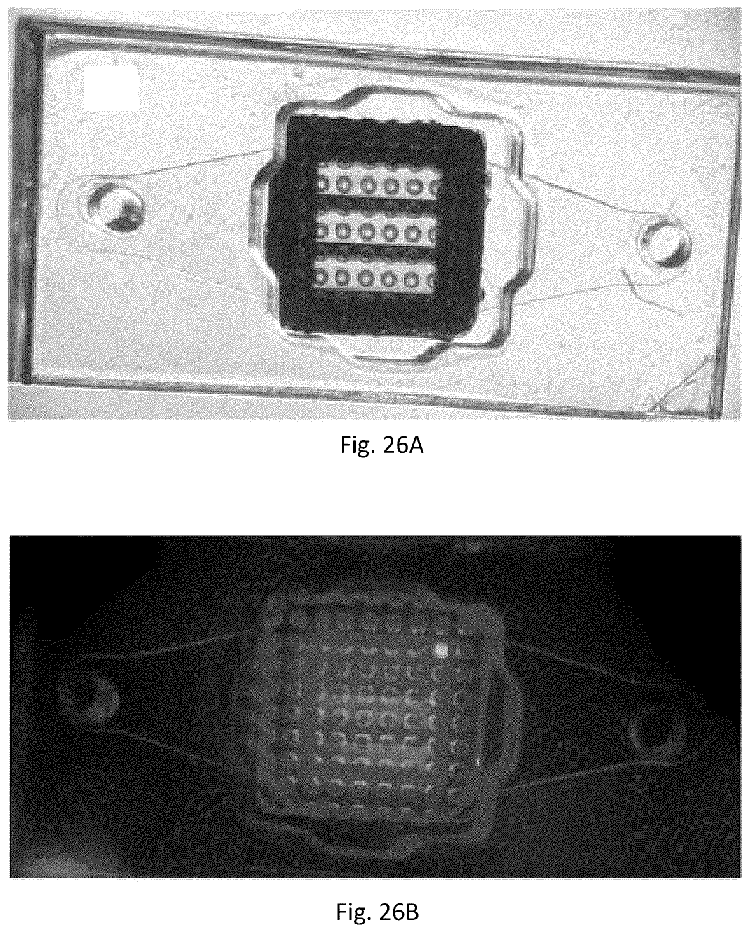

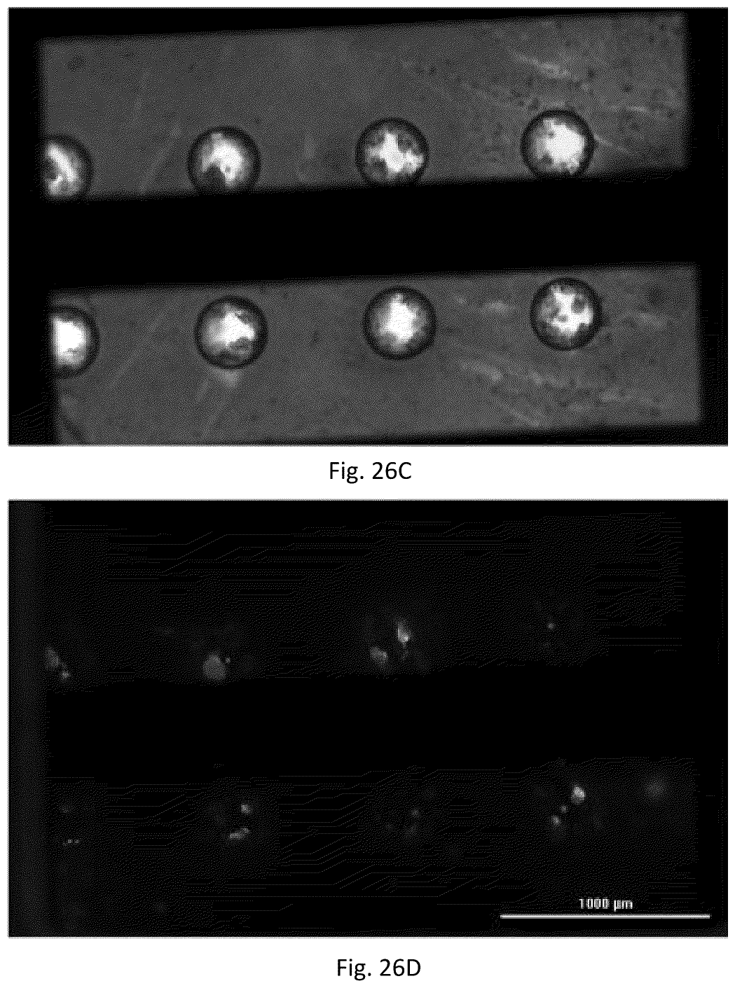

Under a dissection microscope, it is evident when the gap in hybrid MPS device (not the MBs) is filled with hydrogel precursor. In a preliminary test, the gap was filled with PEG hydrogel mixed with fluorescent dextran showing successful filling of the gap (FIGS. 26A and 26B). Successful formation of SGm were confirmed at day 4 in the hybrid μSiM device. FIGS. 26C and 26D shows representative images of fluorescent LIVE (green) and DEAD (red) staining of SGm in MB chips in hybrid μSiM device at Day 4.

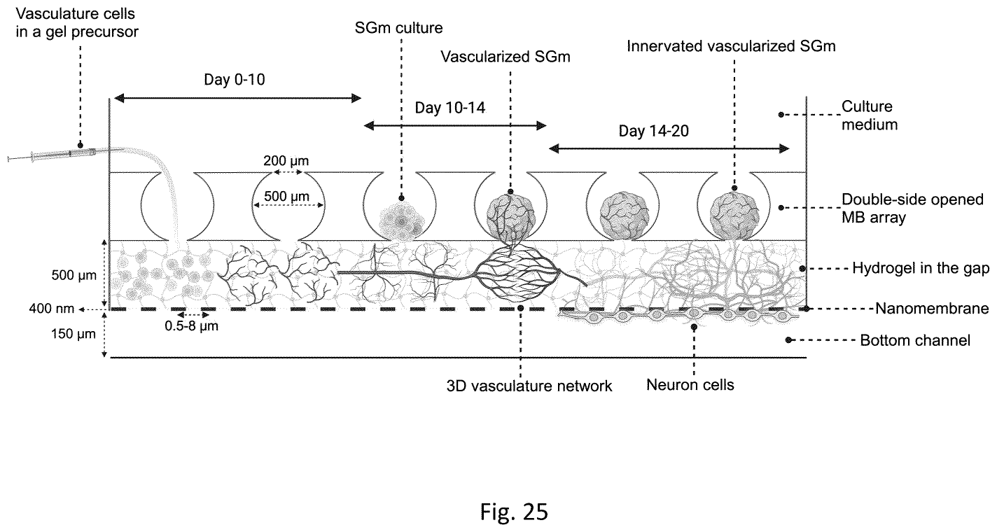

Multi-Organ-on-a-Chip for Innervated and Vascularized Salivary Gland Modeling: This platform is used for the integration of salivary glands tissue mimetics, vasculature and nerve system. Hydrogel precursor is injected into the gap with vasculature cells and the gel (Matrigel and/or collagen type 1) is crosslinked. The hap-hydrogel composition is engineered to support microvascular growth and anastomosis, and it promotes neurite extension. While the cells are forming 3D vasculature network, extracted SG cells were cultured into the MB arrays to form a vascularized spheroid. Nerve cells were also cultured on the underside of the membrane in the bottom channel and the axons project toward the SGm to stimulate them for saliva production. The literature shows that neuron cells may need a distance from the organ to elongate their axons toward the tissue. FIG. 25 shows the development of the vascularized SGm over time. The model can be employed for different organs as autonomic nerve system act on different organs either through blood vessels or directly to the organ e.g. in salivary glands, lung, kidney, or stomach in fight or flight response.

SGm formation in microbubble arrays in the hybrid device over the gel, similar to previous studies in regular MB arrays with single opening, needed to be tested.

FIGS. 26A and 26B demonstrate that the gel has formed in the gap. Red dextran was added to the gel precursor in one of the devices and green microbeads in another device and imaged by fluorescent microscopy. By disturbing the gel, it can be observed that the gel goes back to its initial form demonstrating that the gel is crosslinked and exists in the gap. After confirmation of the existence of gel in the gap, the cell culture was tested with a cell line. It was shown that most of the cells are confined in the microbubbles and a few outside the microbubble and on hydrogel surface. Following the confirmation of cell growth within the microbubbles, mice were sacked and SG cells were extracted and cultured into MB arrays.

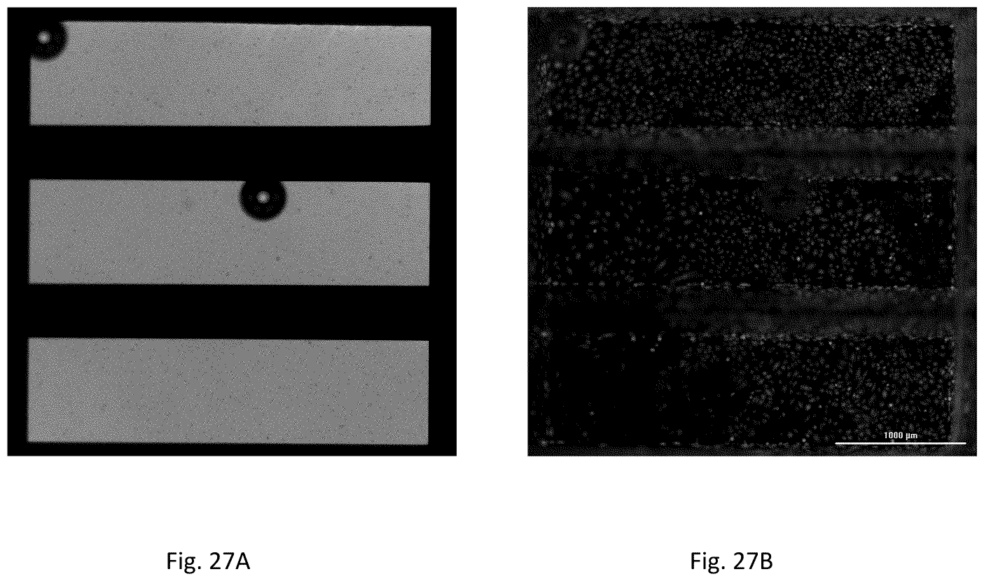

HUVEC Culture on the underside of the nanoporous membrane: HUVEC cells were cultured on the underside of the nanoporous membrane. FIG. 27A depicts a 4× resolution image of Human Umbilical Vein Endothelial Culture (HUVEC) on the underside of the nanoporous membrane at day 4 (Green: Live cells; Red: Dead cells) FIG. 27B depicts a fluorescence microscopy image (×4) of HUVEC culture on the bottom surface of the membrane at day 4 (Green: Live cells; Red: Dead cells).

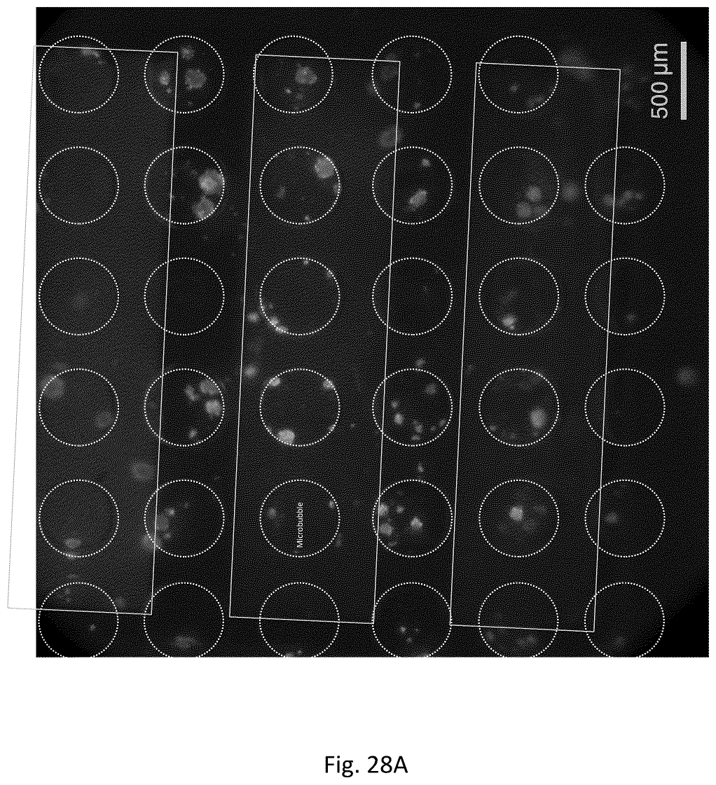

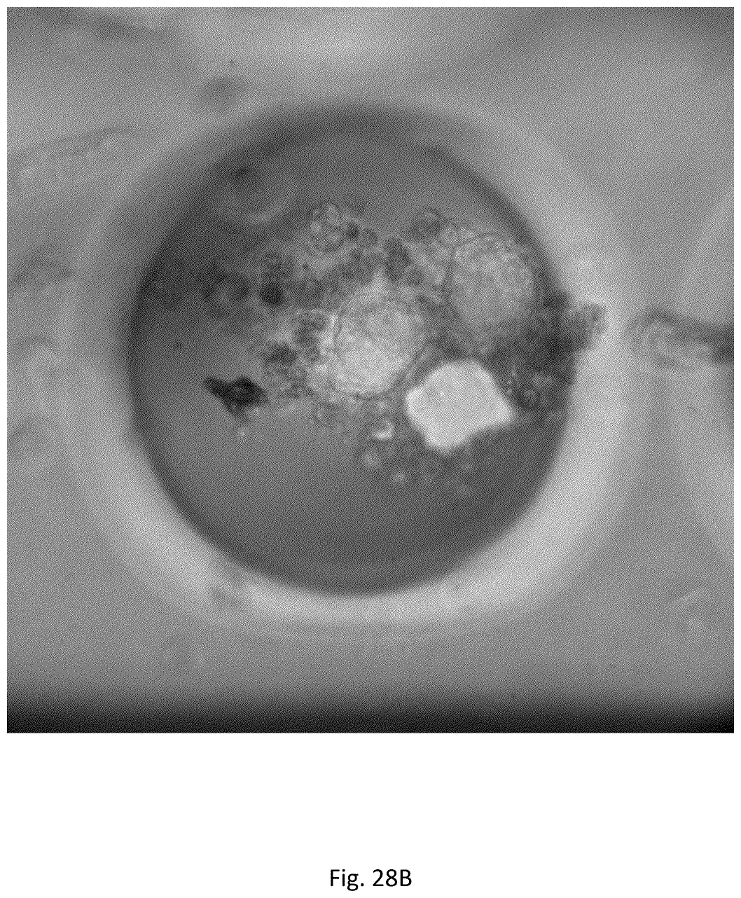

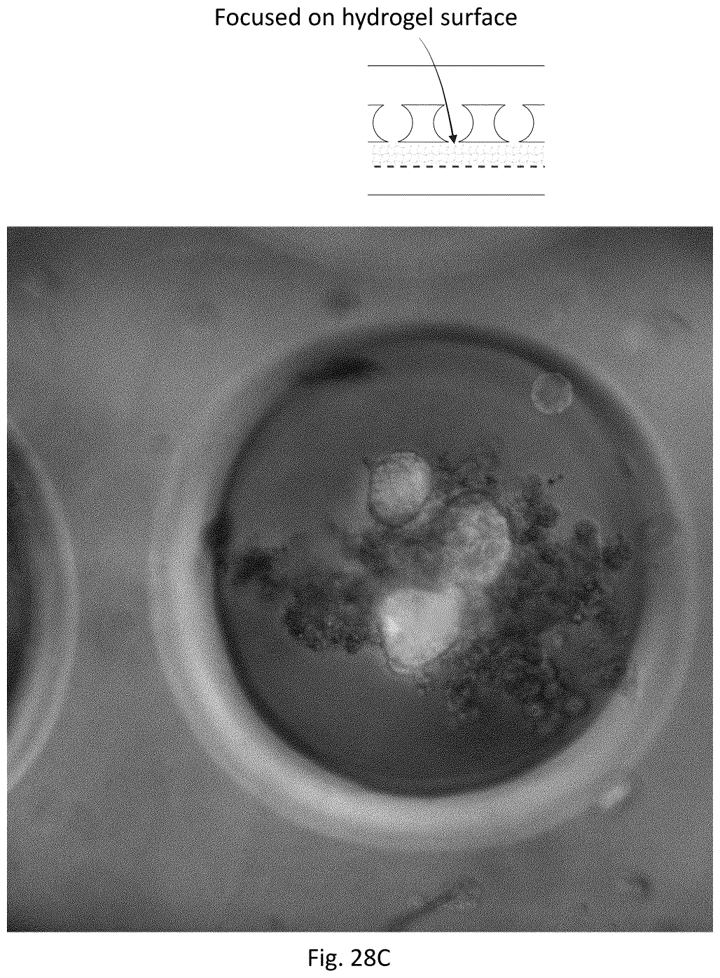

Salivary Gland Culture: SGms were cultured with and without the hydrogels as a bed. FIG. 28A depicts a 10× resolution fluorescence microscopy image of SGm culture in the hybrid device with degradable PEG hydrogel in the gap at day 4 (Green: Live cells; Red: Dead cells). The yellow boxes indicate alignment of the microbubbles with slots in the nanoporous membrane. FIG. 28B depicts a 20× resolution fluorescence microscopy image of SGm culture in the hybrid device with degradable PEG hydrogel in the gap at day 4 (Green: Live cells; Red: Dead cells). FIG. 28C depicts a 20× resolution fluorescence microscopy image of SGm culture in the hybrid device with degradable PEG hydrogel in the gap at day 4 (Green: Live cells; Red: Dead cells).

FIGS. 29A and 29B depict schematics of exemplary MB arrays with small and large bottom openings.

Summary and conclusion: For the first time, SGm were cultured with apical flow in single side open and may replicate in vivo conditions better than static culture. The hybrid μSiM platform can enable multi-organ-on-a-chip (SG with neuron-vasculature system) development. The hybrid μSiM platform has the potential of recapitulating the interaction between three different tissues in various disease models wherein at least one tissue is a multicellular 3D organoid or tissue mimetic. The array enable growth of similar size tissues in an array format for high-throughput drug discovery. The hybrid μSiM enables the combination of different technologies including hydrogel, tissue engineering, and microfluidics. The platform is capable of recapitulating the interaction between three different tissues in various disease models. The modular pSiM utilizes mass-produced components to allow quick assembly of the multi-organ MPS and can enable multi-organ-on-a-chip (SG with neuron/vasculature system) development and serve as a model for various diseases impacting SGs like Sjögren's syndrome, accelerating the development of new therapies.

Technical Advances and Innovations: Microbubble tissue chips support 3D spheroid/organoid long-term growth, phenotype retention, and high-throughput drug screening with defined biomarkers. The hybrid platform enables multi-organ-on-a-chip development with higher in vivo recapitulation, microfluidics, in situ biosensing, high-throughput drug screening with defined biomarkers, personalized medicine and rare disease modeling. Microbubble tissue chips support cell viability and promote organoid formation and enable high-throughput screening. Applications of the microfluidic system may be expanded for different tissues like the retina, lung, and tumor-on-a-chip. The hybrid platform facilitates comprehensive disease modeling by multi-organ development, high-throughput drug screening, and in-situ biosensing in a single microfluidic device and has a huge potential to be employed for different disease models like cancer towards precision medicine.

In some embodiments, the vibratome process is tuned to have the highest control of size and smallest possible second opening in the microbubbles of the MB arrays. In some embodiments, hydrogel formulation is optimized for providing a bed for SGm formation and neurovasculature development. In some embodiments, vasculature and nerve system formation in the hybrid device is performed independently. In some embodiments, selection of proper pore size of the nanomembrane is chosen for the desired application.

The disclosures of each and every patent, patent application, and publication cited herein are hereby each incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A microbubble array comprising:
a microbubble membrane comprising a plurality of microbubbles, each microbubble of the plurality of microbubbles having a first opening and a second opening diametrically opposing the first opening, and
a nanoporous membrane positioned below the microbubble membrane, wherein a first cell type is positioned within at least a portion of the microbubbles of the plurality of microbubbles, and a second cell type is positioned on a bottom surface of the nanoporous membrane.

2. The microbubble array of claim 1, wherein the first cell type comprises at least one of salivary gland tissue cells, salivary gland cell clusters, or salivary gland cells, and the second cell type comprises endothelial cells.

3. The microbubble array of claim 2, further comprising at least a third cell type on the bottom surface of the nanoporous membrane, wherein the third cell type comprises at least one of nerve cells and immune cells.

4. The microbubble array of claim 3, wherein each microbubble of the plurality of microbubbles is at least partially filled with at least one of a polymer, a hydrogel, a polymer crosslinking agent, and a hydrogel crosslinking agent.

5. The microbubble array of claim 4, further comprising a spacing portion with a central opening positioned between the microbubble membrane and nanoporous membrane, wherein a gap with a volume is formed by the opening and the gap is at least partially filled with a polymer or hydrogel.

6. The microbubble array of claim 5, wherein the first opening of each microbubble of the plurality of microbubbles is larger than the second opening.

7. The microbubble array of claim 6, wherein the plurality of microbubbles has a microbubble density of 40 to 30,000 MB/cm².

8. The microbubble array of claim 7, wherein the microbubble membrane is formed from polydimethylsiloxane (PDMS), and the nanoporous membrane is formed from silicon.

9. The microbubble array of claim 8, wherein each microbubble of the plurality of microbubbles has a diameter ranging between 20 microns and 2000 microns, and wherein the first opening and the second opening of each microbubble of the plurality of microbubbles has a width ranging between 5 microns and 1000 microns.

10. The microbubble array of claim 9, wherein the thickness of the microbubble membrane ranges between 0.03 mm and 3 mm, and the thickness of the nanoporous membrane ranges between 50 nm and 500 nm.

11. A microfluidic device, comprising:
a housing at least partially enclosing at least one microbubble array of claim 1;
a top channel in the housing fluidly connected to the first opening of each microbubble of the plurality of microbubbles; and
a bottom channel in the housing fluidly connected through the pores of the nanoporous membrane to the second opening of each microbubble in the plurality of microbubbles, wherein one or more fluids may be flowed into the top channel and collected or analyzed from the bottom channel.

12. The microfluidic device of claim 11, further comprising at least one sensor positioned below the nanoporous membrane in the bottom channel, configured to assess cell secreted factors.

13. The microfluidic device of claim 11, further comprising at least one of a sensor, optical sensor, and photonic array configured to optically interrogate each microbubble of the plurality of microbubbles.

14. The microfluidic device of claim 11, further comprising at least one sensor coupled to the microbubble membrane, configured to measure the electrical resistance of the microbubble membrane.

15. The microfluidic device of claim 15, wherein the one or more fluids comprise at least one of cell culture media, water, blood, solutions, solutions of drugs, solutions of therapeutics, solutions of bioactive substances, solutions of bioactive compounds, buffers, stimulants, and combinations thereof.

16. A microphysiological system, comprising:
a top portion having a body with at least one cavity, an inlet, and an outlet, each passing through the body;
at least one microbubble array of claim 1 positioned within the at least one cavity;
a bottom portion comprising a plate having an indentation forming a channel, wherein the top portion is fixedly and removably attached to the bottom portion and the cavity, inlet and outlet are fluidly connected by the channel, and wherein one or more fluids may be flowed into the inlet, analyzed within the channel, and collected from the outlet.

17. The microphysiological system of claim 16, wherein the microbubble membrane is attached to the nanoporous membrane with a spacing portion forming a gap between the microbubble membrane and the nanoporous membrane.

18. The microphysiological system of claim 17, wherein the spacing portion comprises a layer of pressure sensitive adhesive with a central opening passing through the layer, and wherein the gap is at least partially fluidly filled with a hydrogel.

19. The microphysiological system of claim 18, wherein the hydrogel comprises at least one PEG, Matrigel, collagen, fibrinogen, alginate, polyacrylamide, and hyaluronic acid.

20. The microphysiological system of claim 19, further comprising one or more sensors positioned within the top or bottom portion of the system, wherein the one or more sensors are selected from the group consisting of: optical sensors, photonic arrays, transelectrical resistance (TEER) sensors, biosensors, flow rate sensors, fluid pressure sensors, temperature sensors, pH sensors, oxygen sensors, carbon dioxide sensors, and electrochemical sensors.

21. The microphysiological system of claim 20, wherein the one or more fluids comprise at least one of cell culture media, water, blood, solutions, solutions of drugs, solutions of therapeutics, solutions of bioactive substances, buffers, stimulants, or combinations thereof.

22. The microphysiological system of claim 21, wherein the system is configured to recapitulate one or more disease states, wherein the disease states are selected from tumors, eye diseases, lung diseases, kidney diseases, or stomach diseases.
